(19) United States
(12) Patent Application Publication
(10) Pub. No.: US 2020/0163607 A1
(43) Pub. Date: May 28, 2020

(54) METHOD AND SYSTEM FOR ACCURATE DESCRIPTION OF PERSONALITY TYPES AND BEHAVIOR

(71) Applicant: YOUSEF AL-HASHASH, SAFAT (KW)

(72) Inventor: YOUSEF AL-HASHASH, SAFAT (KW)

(21) Appl. No.: 16/695,714

(22) Filed: Nov. 26, 2019

Related U.S. Application Data
(63) Continuation of application No. 14/168,361, filed on Jan. 30, 2014, now abandoned.

(51) Int. Cl. A61B 5/16; G16H 10/20; G09B 5/00

(52) U.S. Cl. CPC A61B 5/167; G09B 5/00; G16H 10/20

(57) ABSTRACT

A method for assessing an individual's personality type and behavior contemplates the following steps. Entering four cognitive functions, Sensing (S), Feeling (F), Thinking (T) and Intuitive (N) into a computer and separating each cognitive function into eight modified functions wherein each total ES and EI=100%. Each of the cognitive functions are analyzed for judging and perception to create 16 modified functions. Then five models are established for each of the modified functions. An individual and/or their behavior preferences with respect to the 16 modified functions and each of the five models for each modified functions are evaluated and/or analyzed.

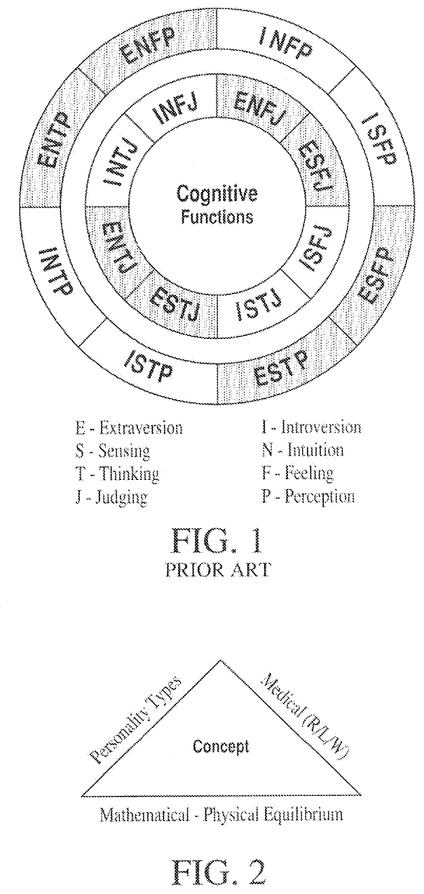

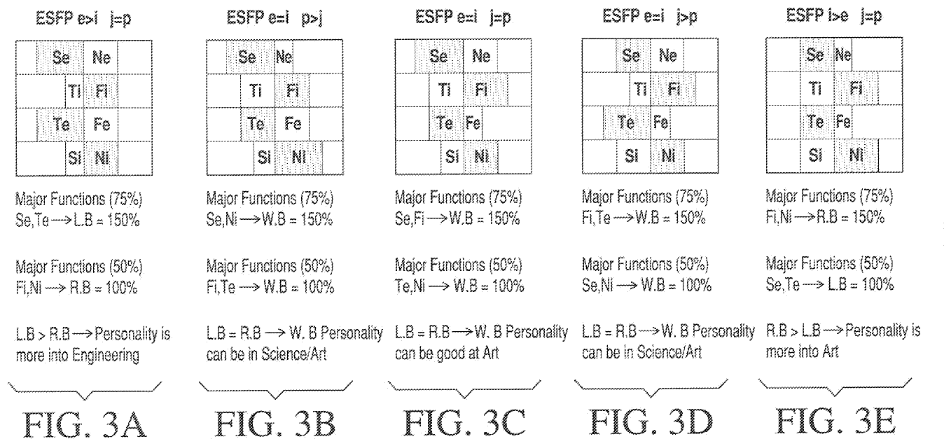

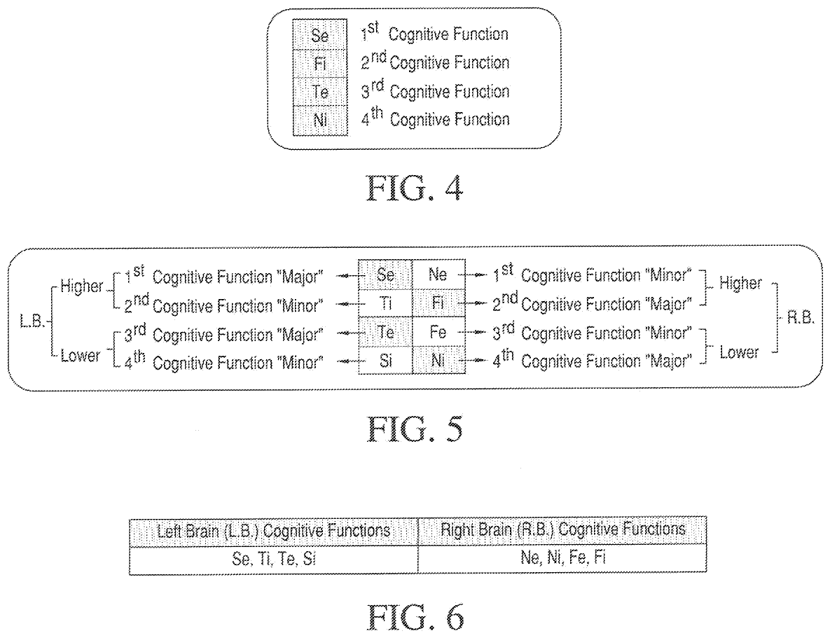

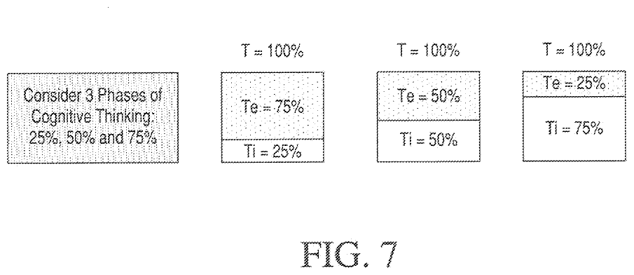

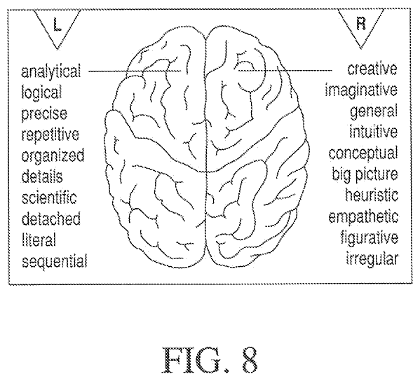

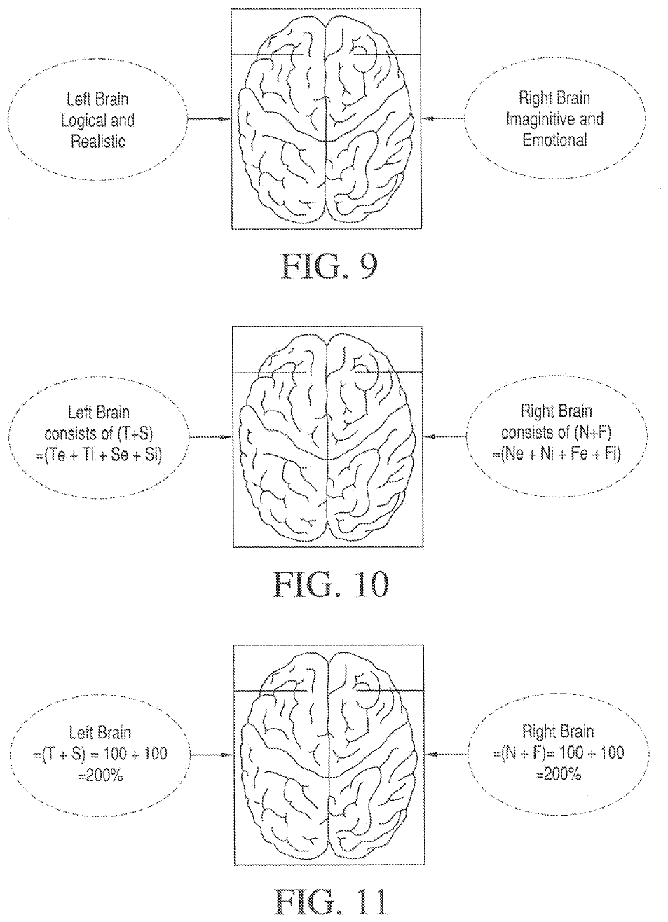

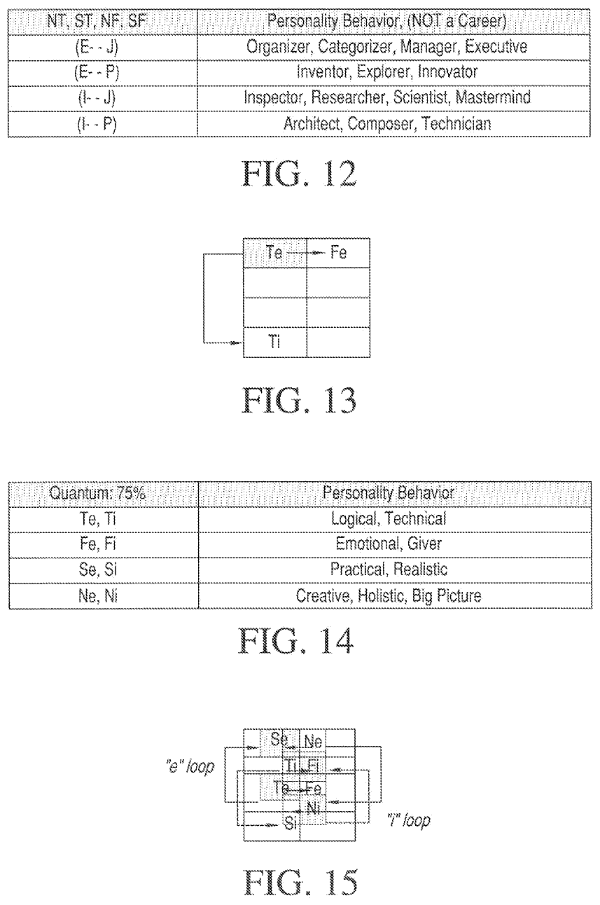

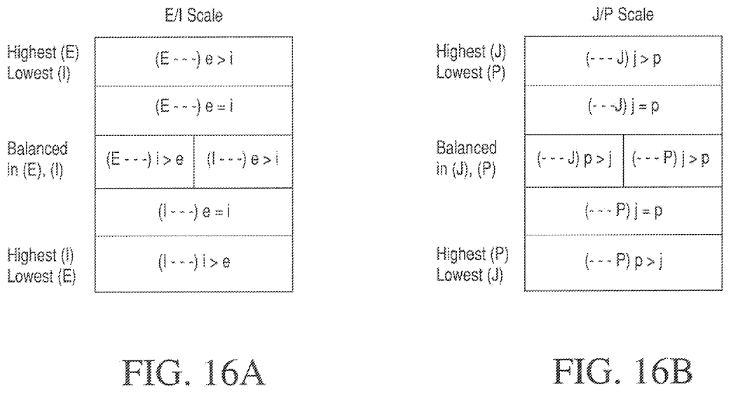

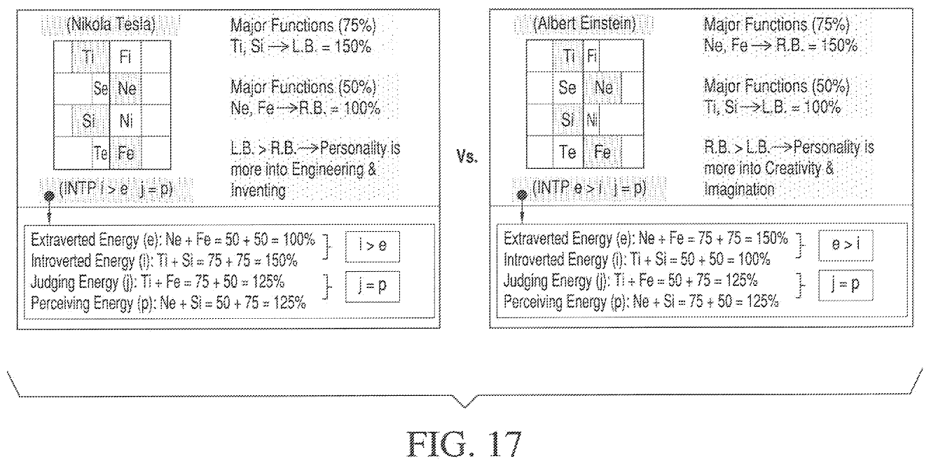

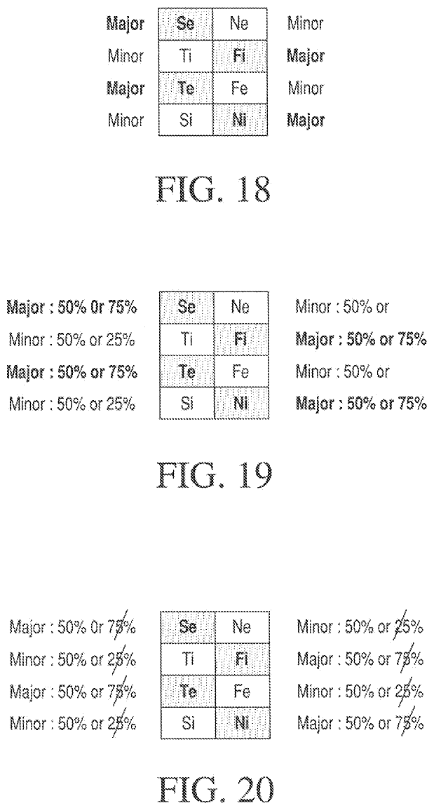

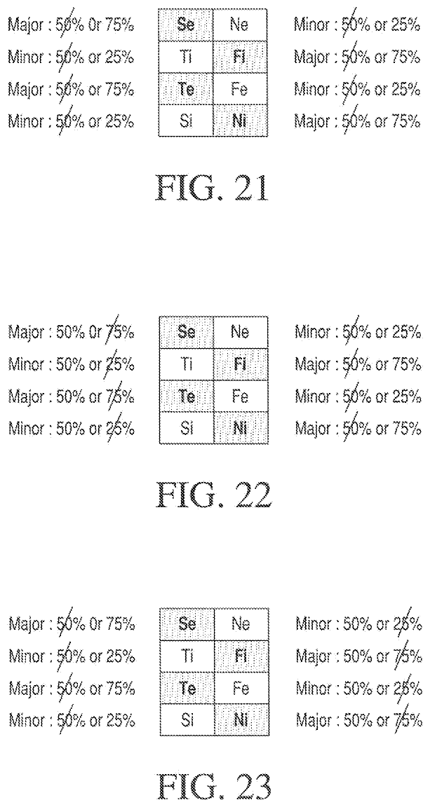

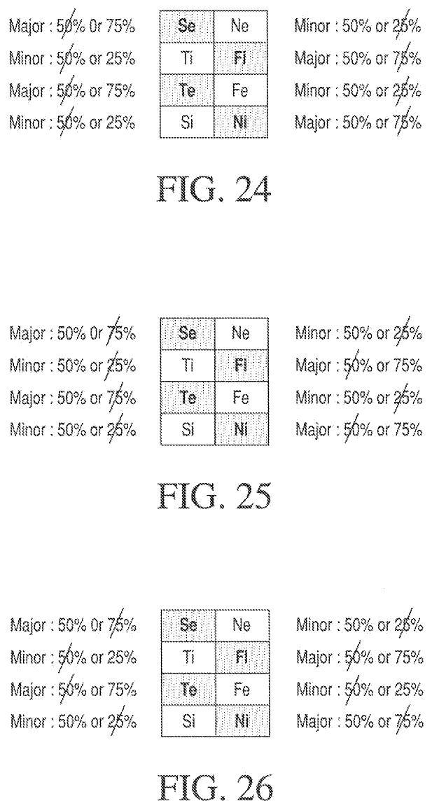

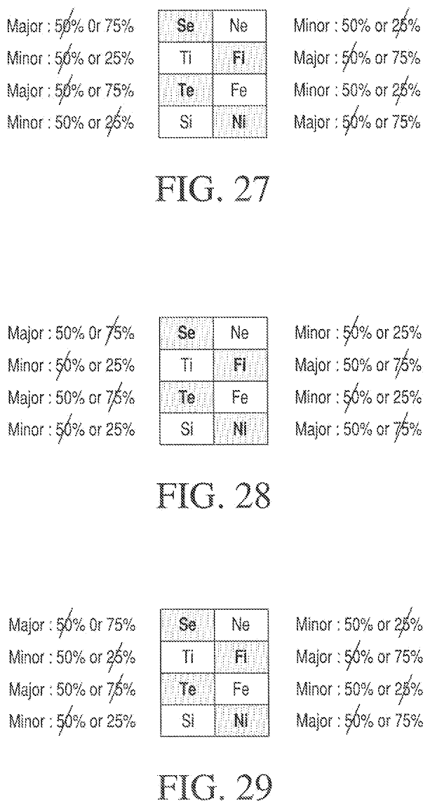

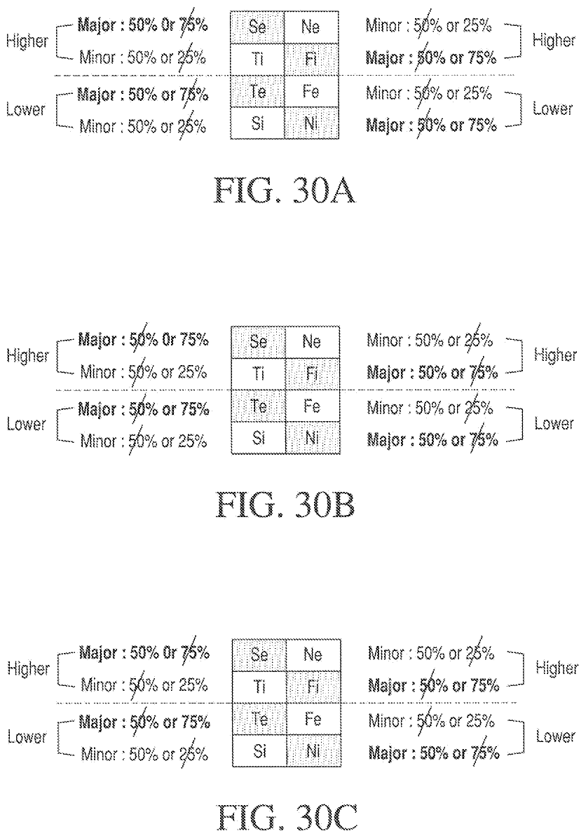

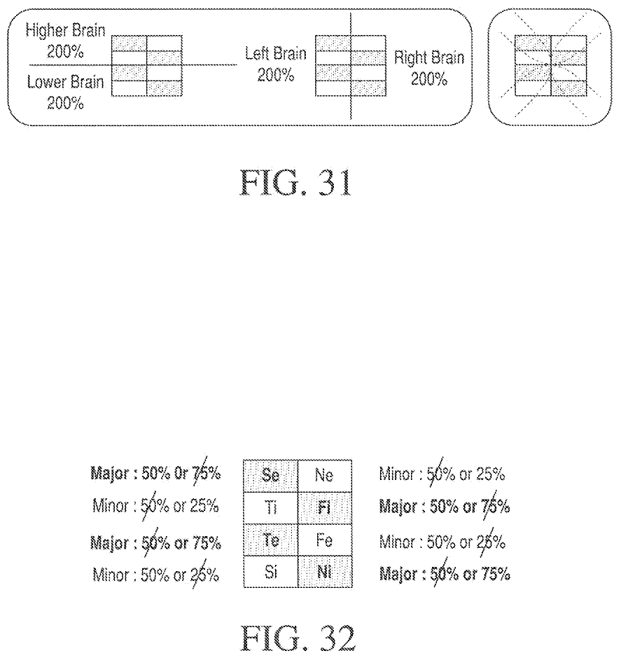

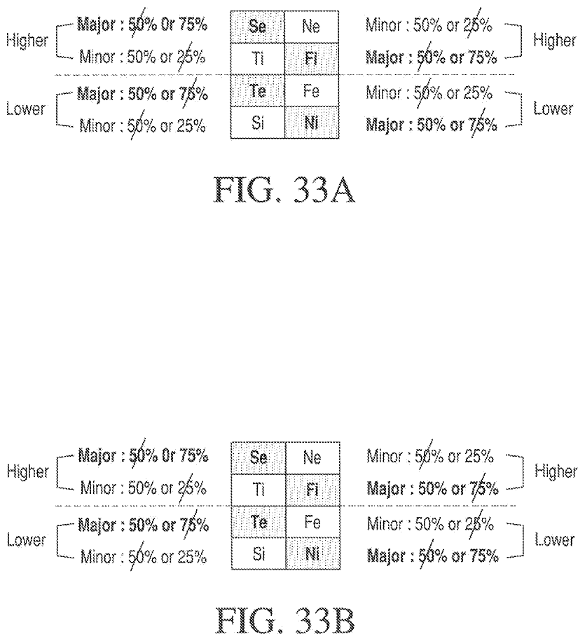

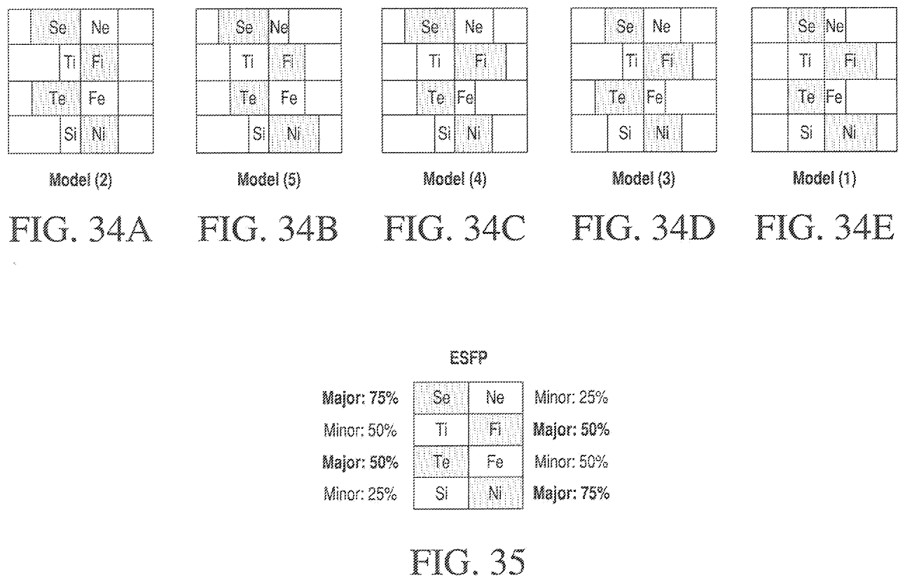

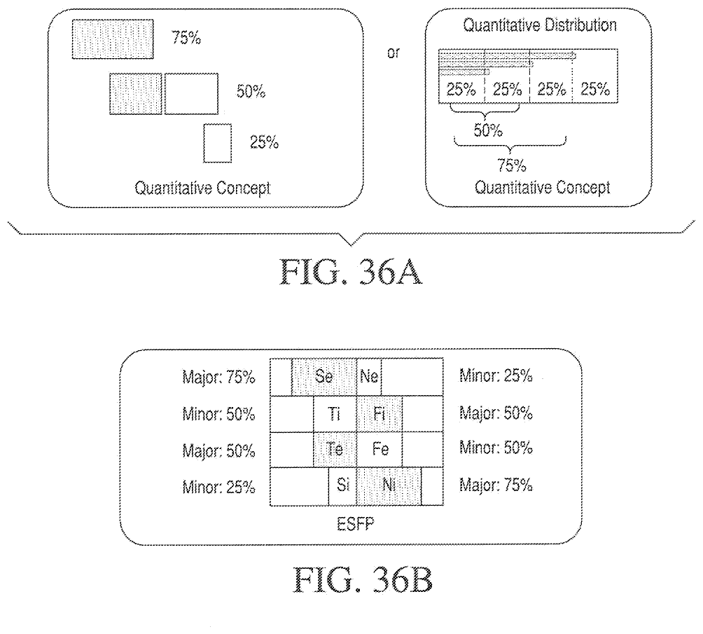

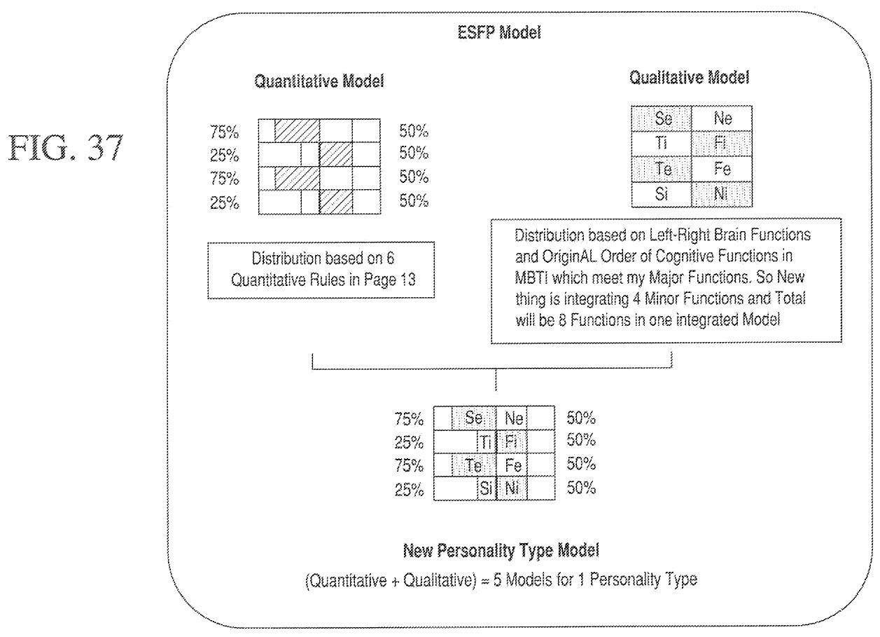

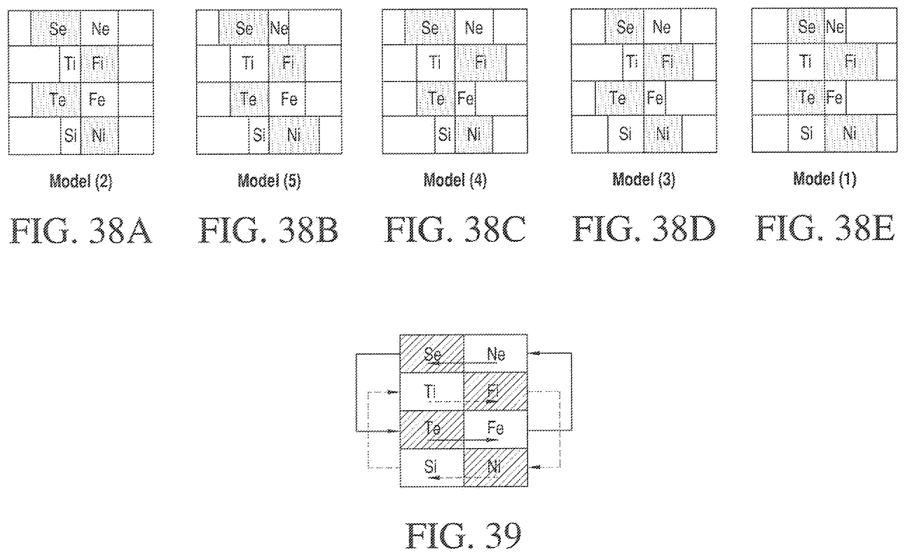

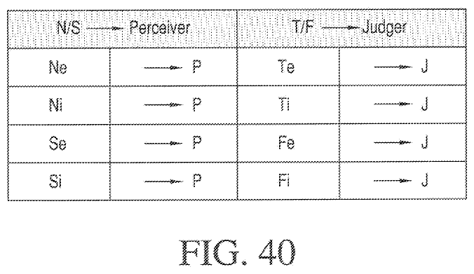

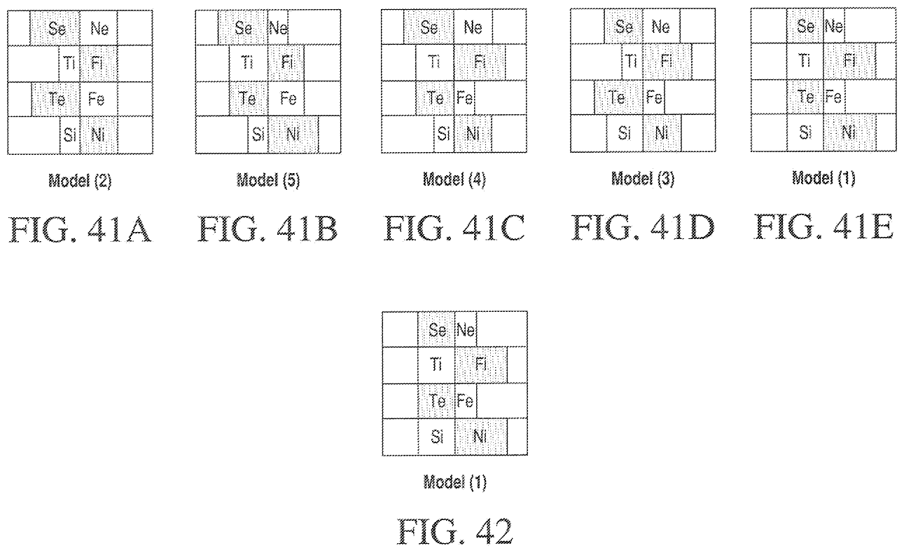

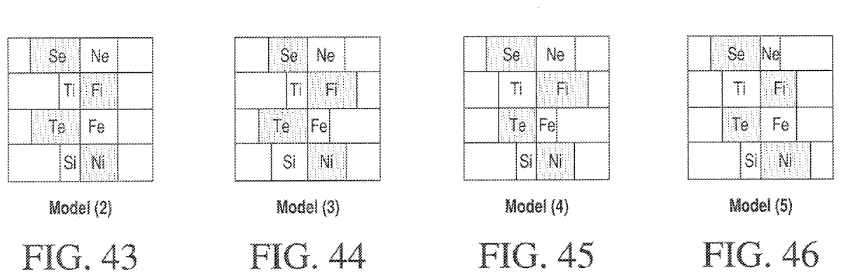

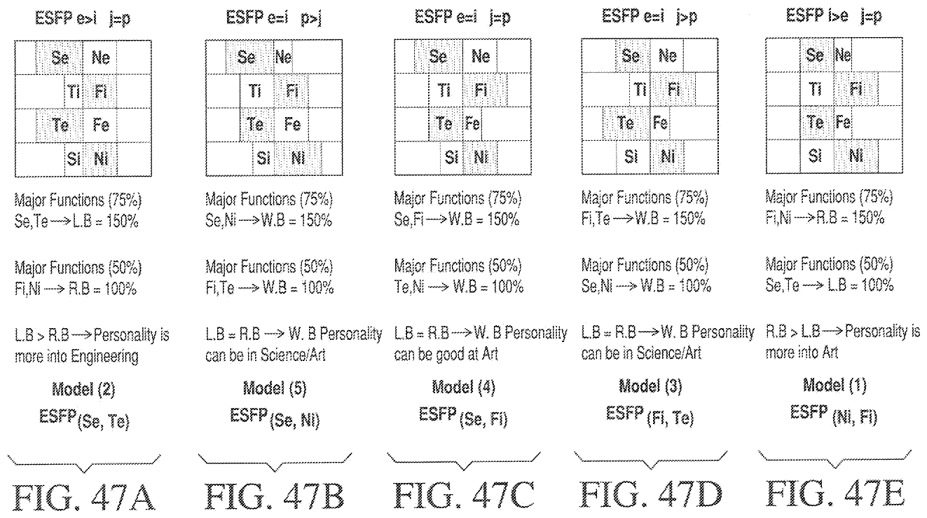

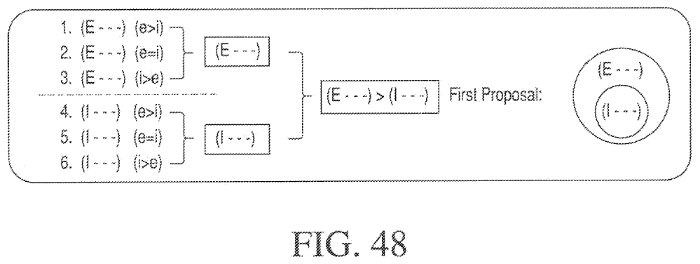

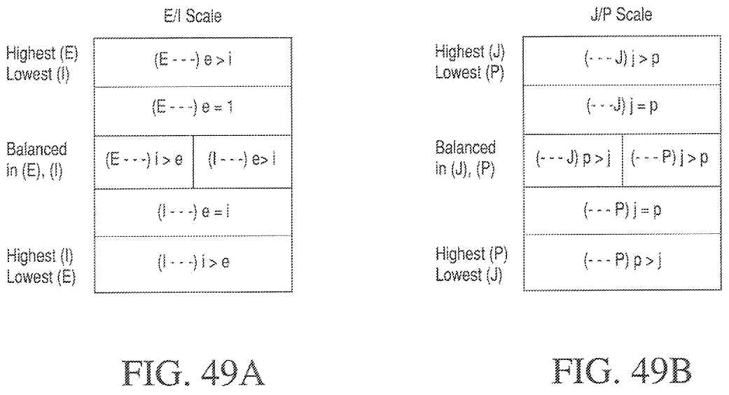

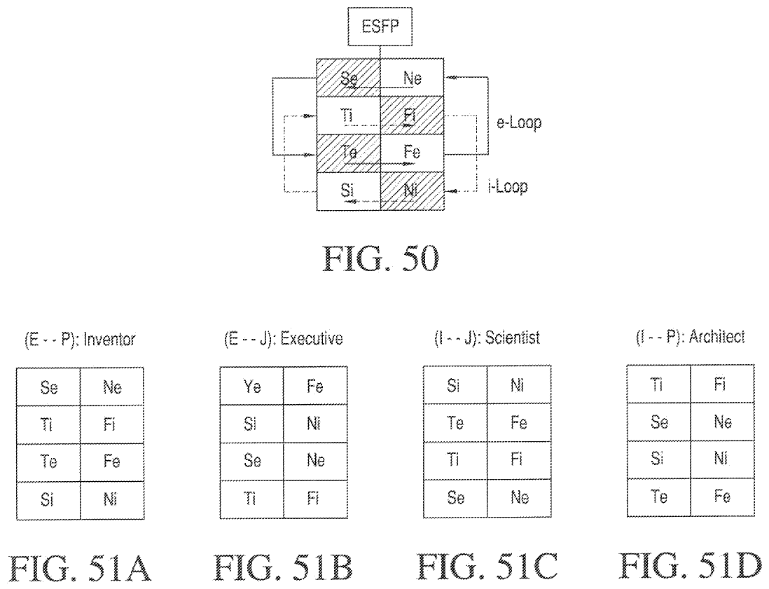

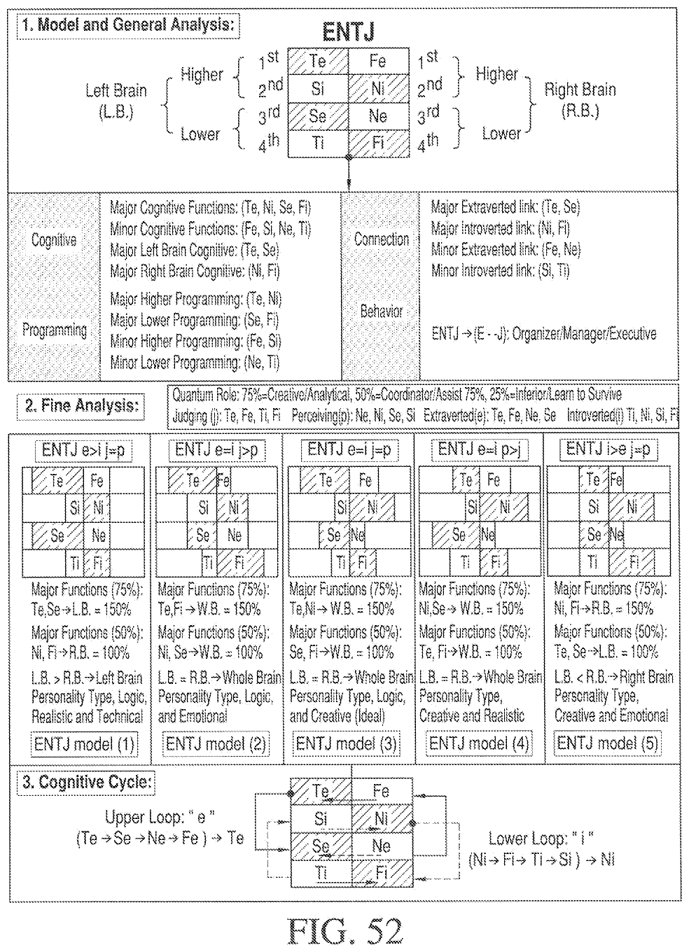

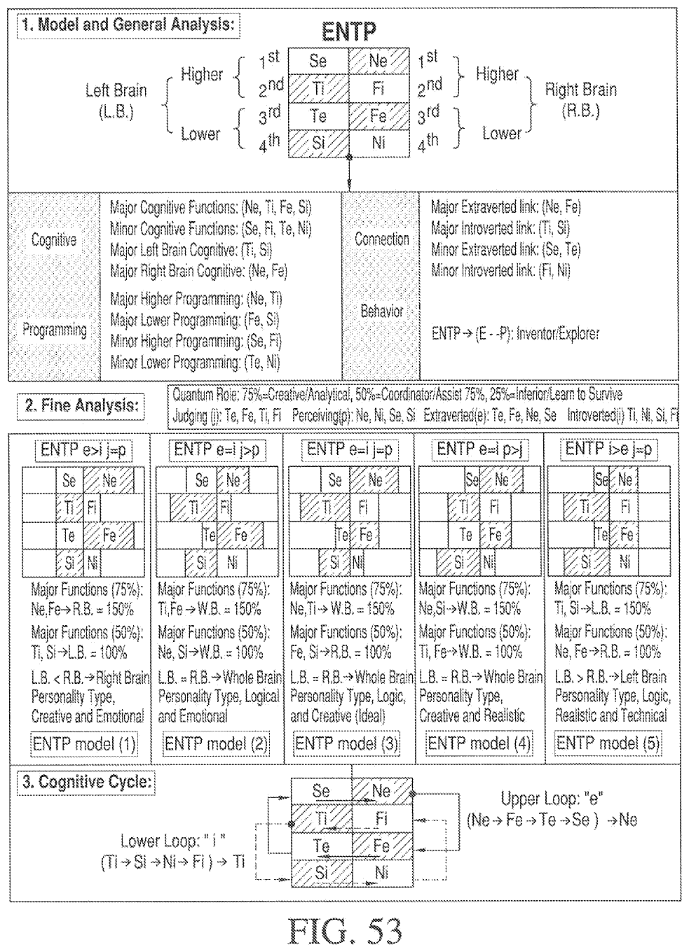

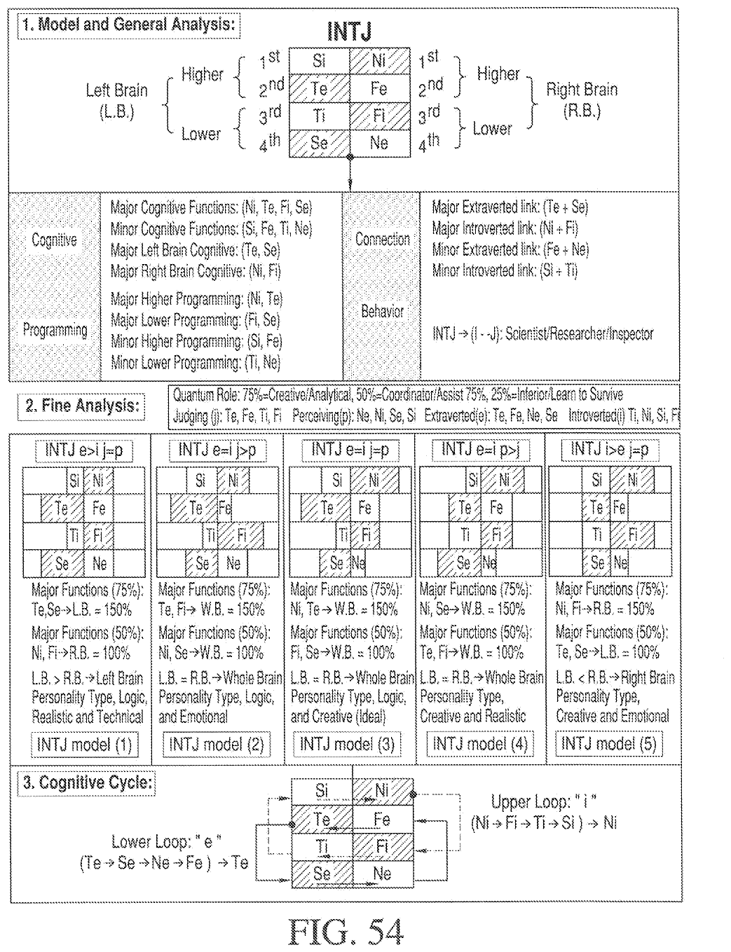

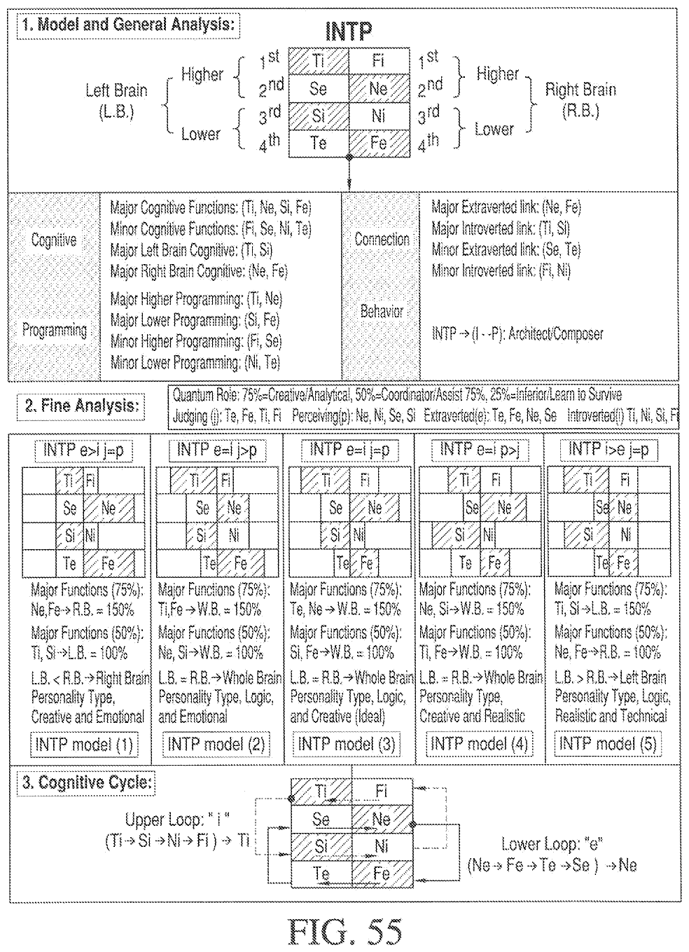

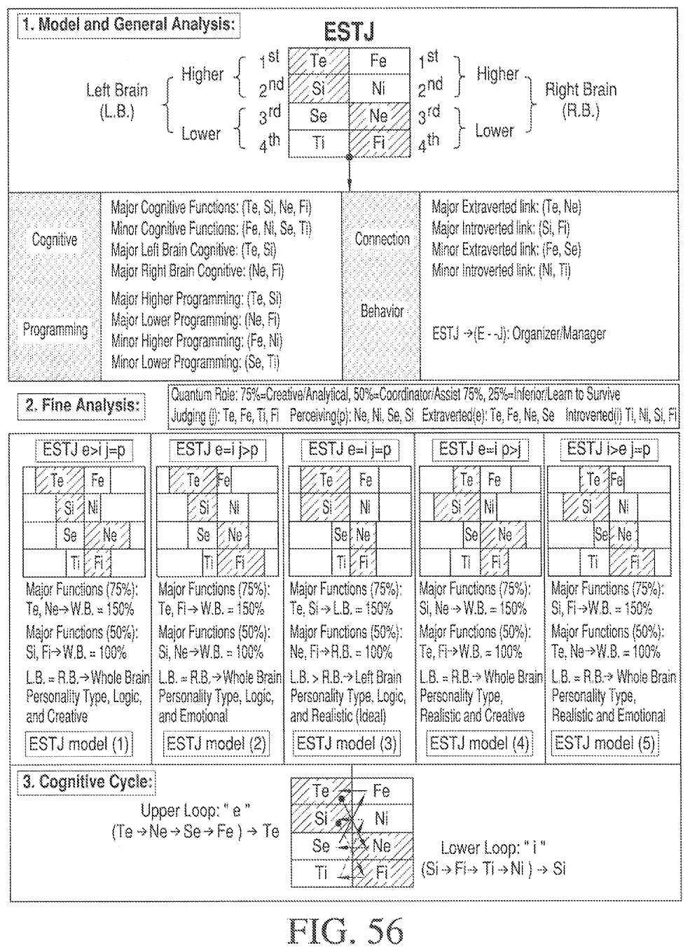

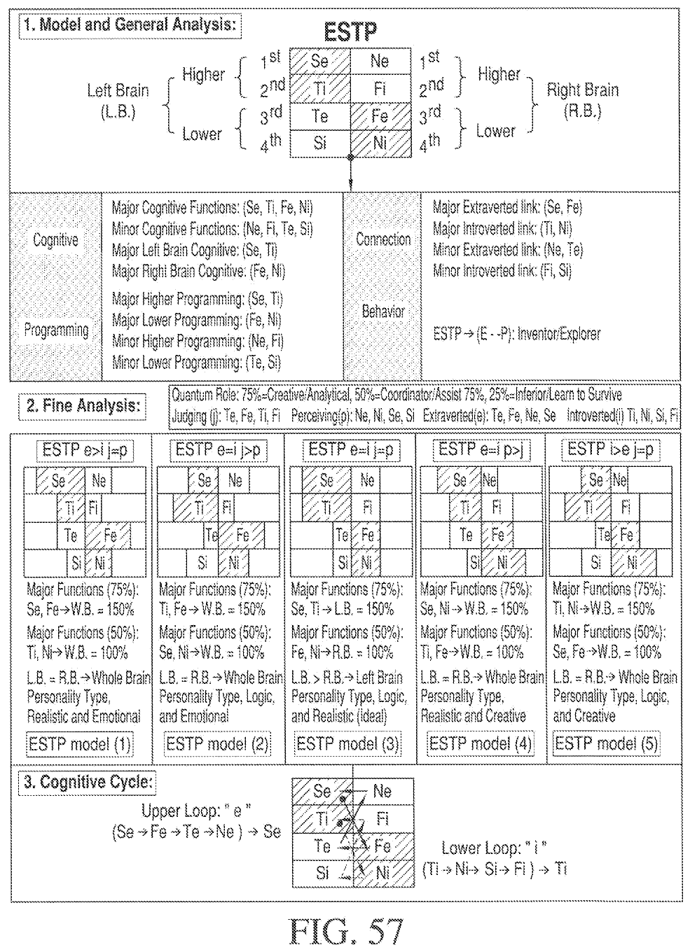

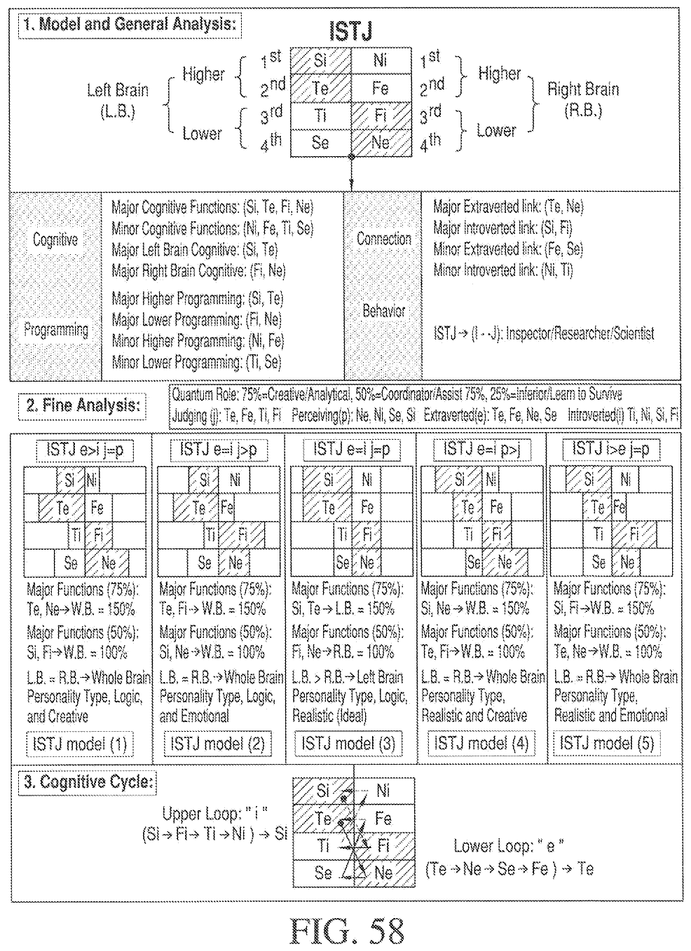

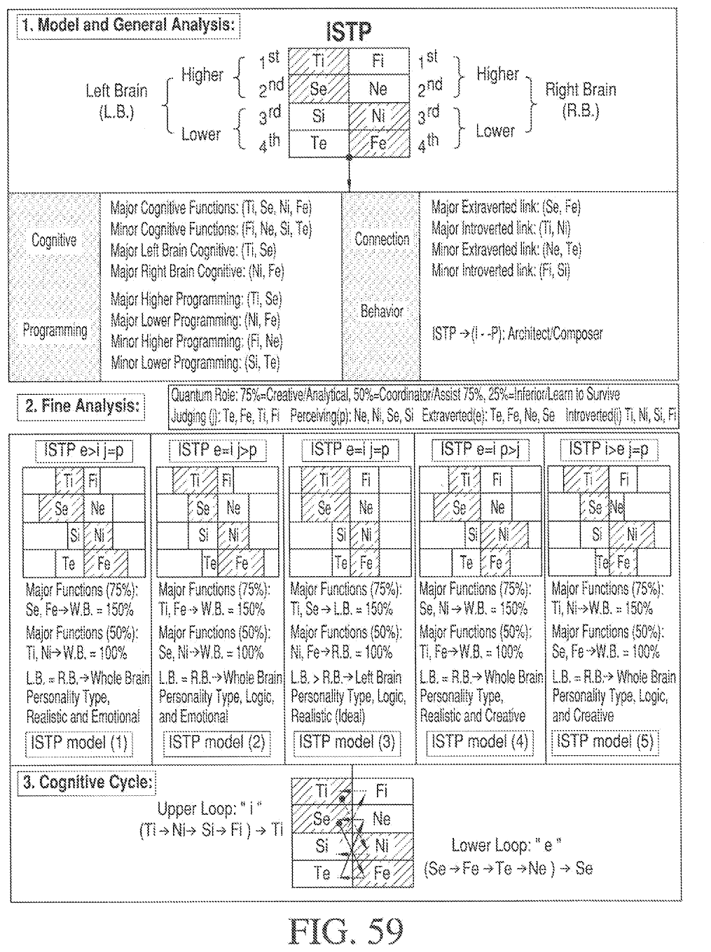

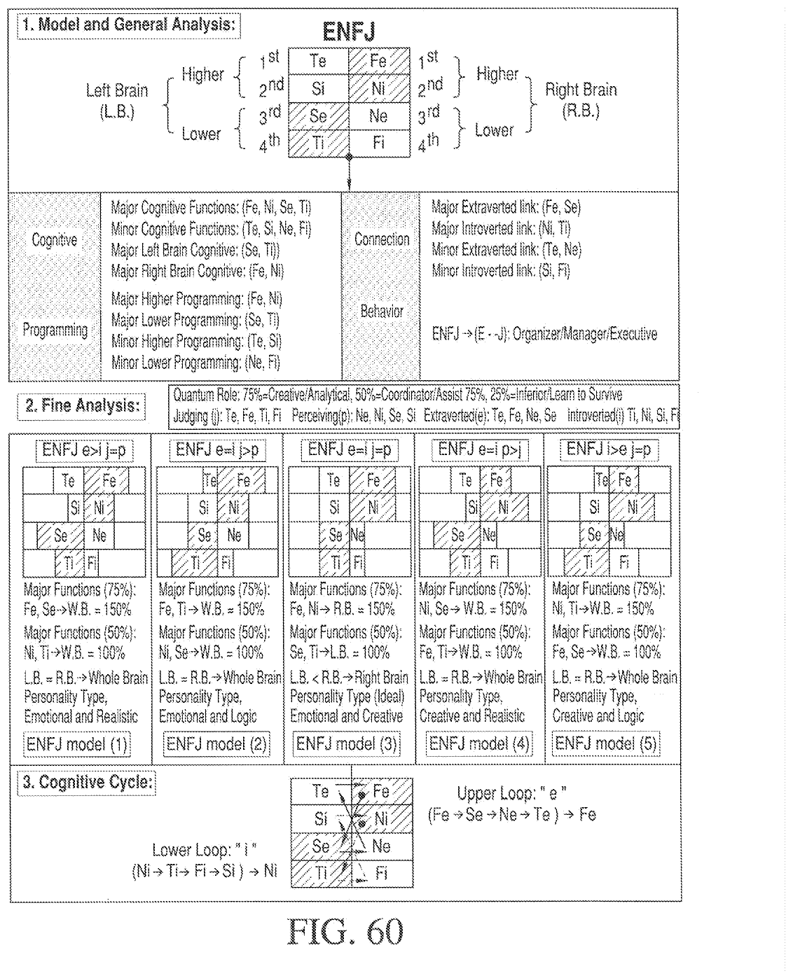

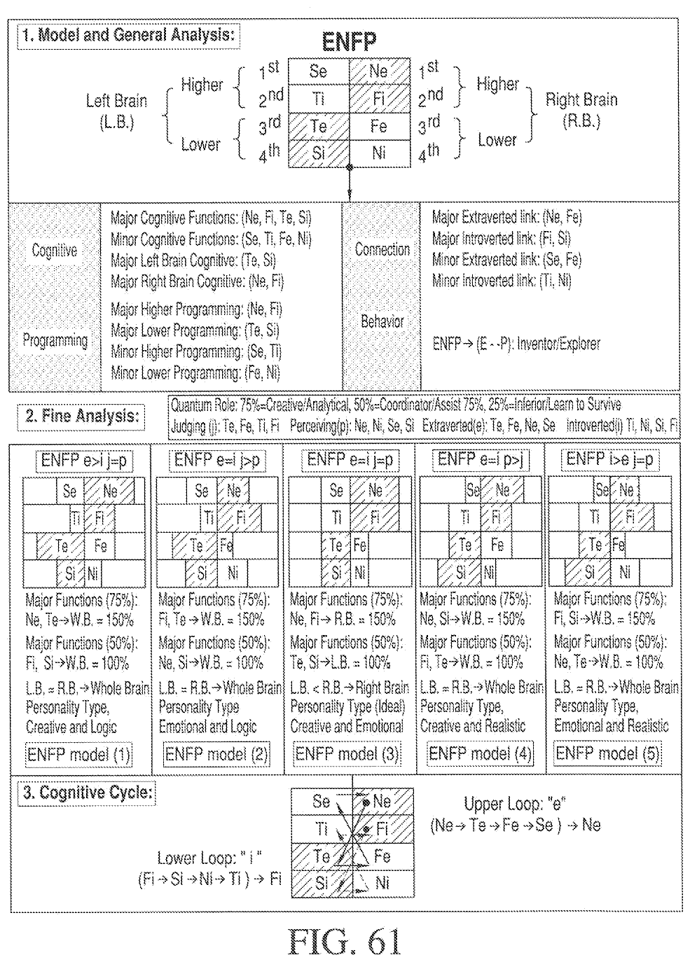

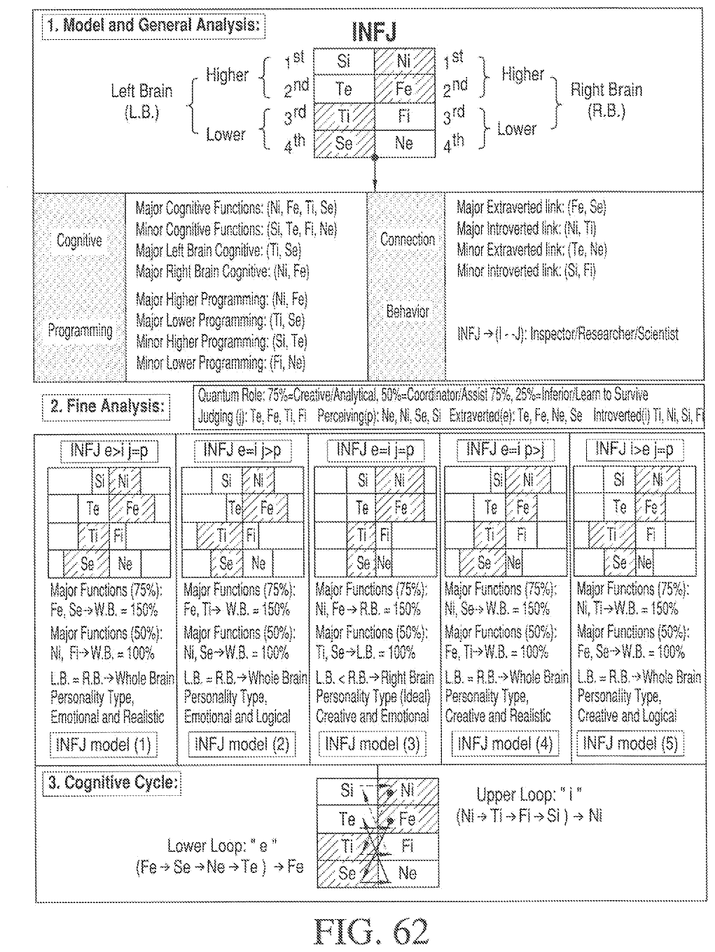

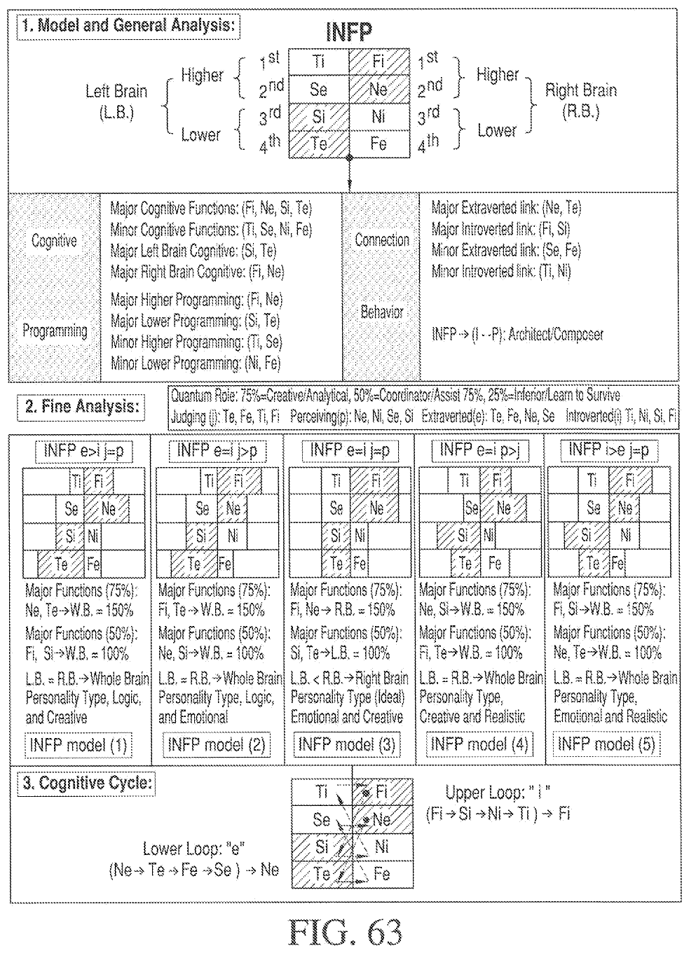

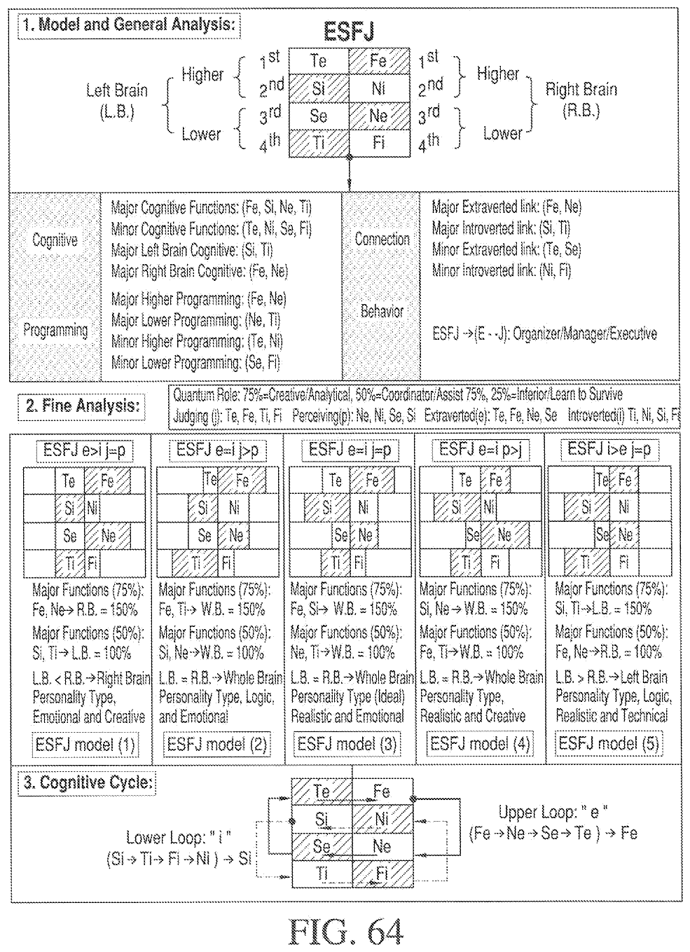

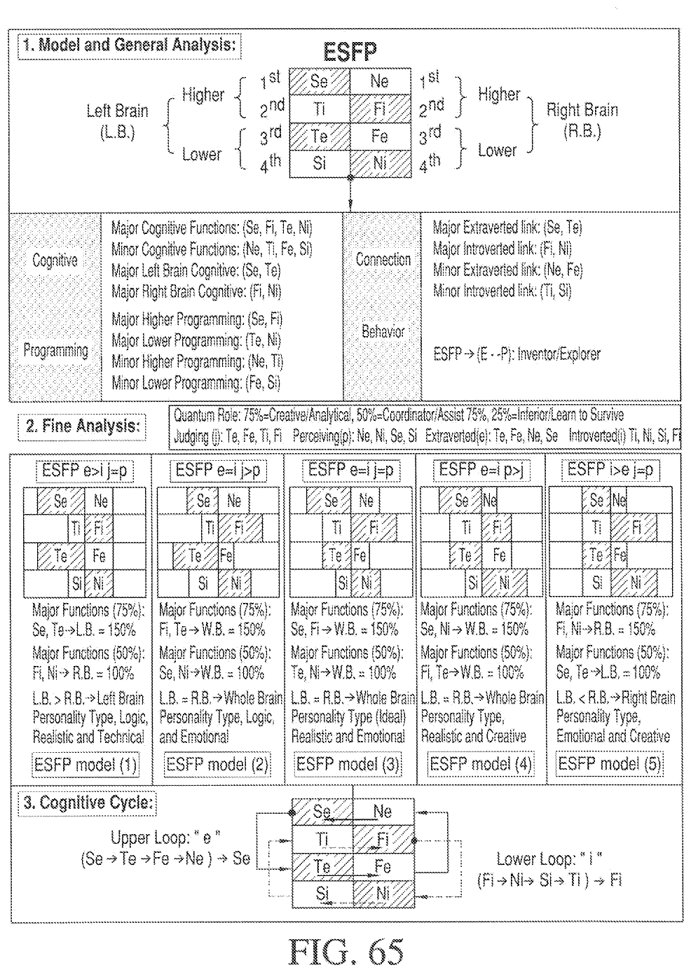

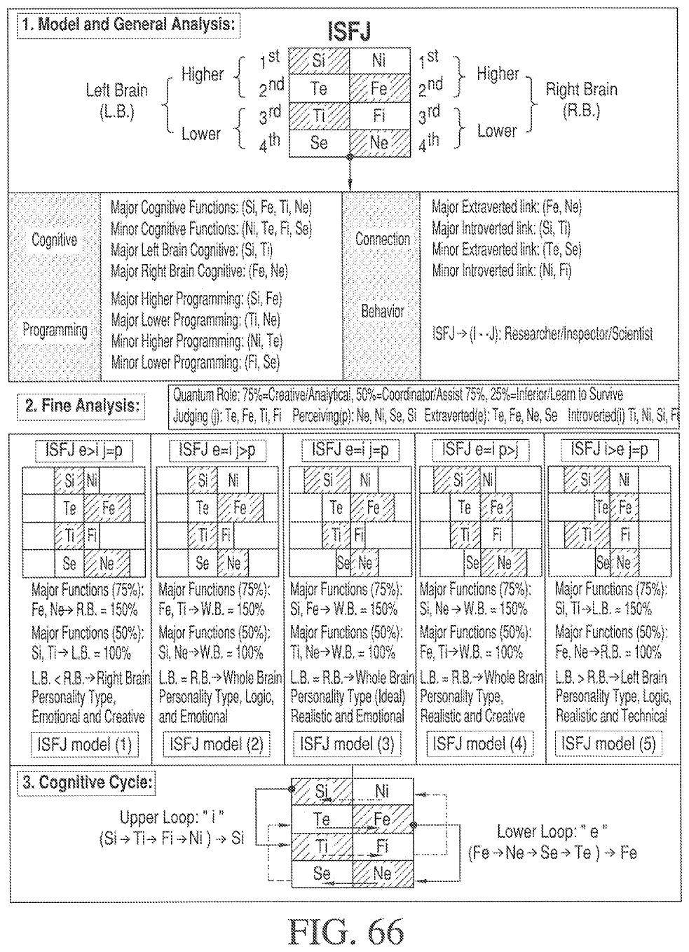

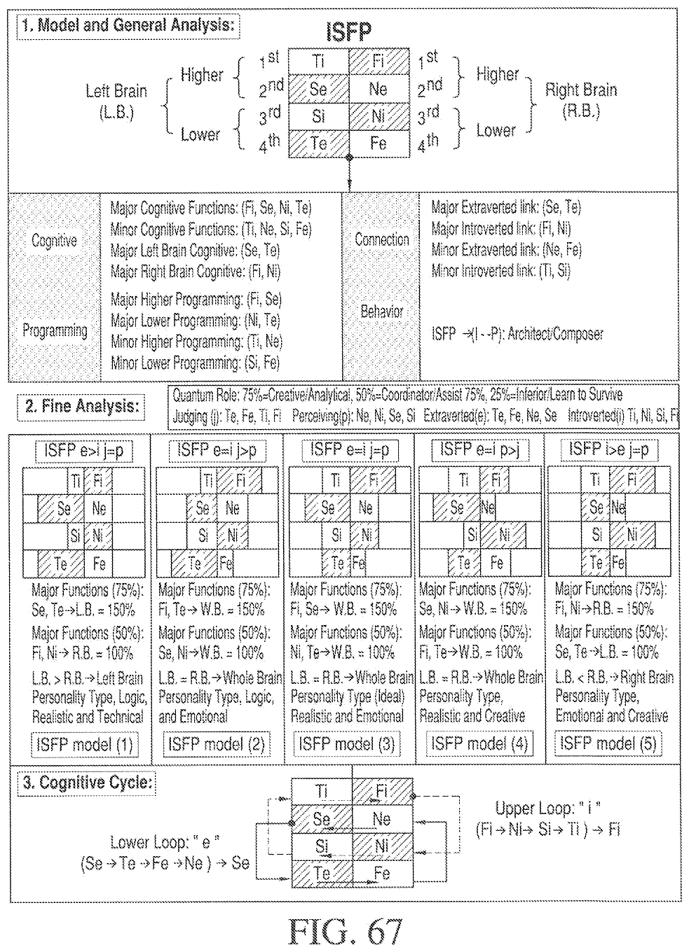

### DETAILED DESCRIPTION OF THE PREFERRED EMBODIMENTS OF THE INVENTION

The present invention is based on prior art personality types as for example Meyers-Briggs Type Indicator (MBTI). In the present invention, Applicant divided each of the 16 personality types into five models. These five models provide a better understanding in defining personality types as an indicator. What Applicant has done is to develop a method for applying quantum energies of the eight cognitive functions “Jungian Functions,” applied on the 16 personality types in a mathematical-physical model. To be more specific, the following models illustrate these concepts.

An example that explains the concept of the present invention is an illustration by a Comparison for Current (ESFP) Personality Type Model and (ESFP) Models:

The Current (ESFP) Model is:

The New Personality Type Model for (ESFP) is then:

The 1^{st} Difference is: in Applicant's Functions (All Functions) are in one Integrated Model. The Major/Essential Functions in (ESFP) Model in order are: (Se, Fi, Te, Ni) and The Minor/Assist Functions in (ESFP) model in order are: (Ne, Ti, Fe, Si). The prior model is only including 4 Cognitive Function: (Sc, Fi, Te, Ni).

The 2^{nd} Difference is: Applicant's model is distributed based on Right/Left Brain Functions. The Right Brain (R.B) is more Intuitive, Creative, Big-Picture, Holistic and Emotional where the Left Brain (L.B) is more Logical, Practical, Realistic and Technical. See the Vertical Lines which divides Right Brain (R.B) on your Right Hand and Left Brain (L.B) on your Left Hand. The prior model of MBTI didn't interpreter Right/Left Brain Behavior of a Personality Type. In a Quantitative Concept consider why some of (ESFP)s can be Engineers where other (ESFP)s can be Artists. A Reference to Right/Left Brain Functions can be found at:

http://pamela99.hubpages.con/hub/Right-Brain-VS-Left-Brain-Functions

Applicant divided the 8 Cognitive Functions into Right and Left Brain functions as a Rule for All Personality Types but the order of 8-Functions will differentiate from Personality to another.

A reference for 8-Cognitive Functions or Jungian Functions (Carl Jung Functions) can be found at:

http://www.cognitiveprocesses.com/Cognitive-Functions/index.cfn

The 3^{rd} Difference is: Programming of Higher Thinking and Lower Thinking. In Prior Art; MBTI, (Se, Fi) are main Functions; say Higher Thinking. In Applicant's Concept (Se, Ne, Fi, and Ti) are All Higher Thinking Functions.

1. Personality Types: where 8 Cognitive Functions Play the main Role in Establishing a New and Accurate Personality Type Model. Applicant has Four Major Functions form the main or Front Personality Type and Four Minor Functions which could be the Assistant or the Behind of Personality Type”. In another way of Explanation; Applicant tests are easy to use Major Functions or they are Automatically used (as By default). But there is some resistance by using Minor Functions. Applicant's Concept doesn't conflict with Personality Types Researchers such as; Linda V. Berens. She added four additional functions called “Shadow” Functions (See Shadow Functions for INTJ as an Example in: http://en.wikipedia.org/wiki/INTJ). The Difference in Applicant's Concept is: Applicant used The Eight Cognitive Functions (All Cognitive Functions) integrated in one model. All Cognitive Functions work in a hannonious system that can appear in Normal or tinder stress Situations.

2. The Golden Ratio (Phi) or (1.66=ϕ): Which was discovered in Fibonacci Series by Mathematician Fibonacci (http://en.wikipedia.org/wiki/Fibonacci). This Ratio applied in many fields in life: Cosmology, Art, Architecture, Mathematics (http://mathworld.wolfram.com/GoldenRatio.html) and All Human Body (Face, Hand, Leg, etc). For Example: if you divide the length of human face on its width, the result will give you 1.6. The same thing if you divide the length of your arm versus the length of your hand, the result is 1.6 too. The Smart Connection here is the number 1.6 to Applicant's new Personality Type Concept or say to Psychometrics Field). The Inspiration comes from the Number of Personality Type 16. Now to apply the Golden Ratio to Applicant's Concept, it will be like the following: “When you divide the Total Quantum of the Four Major Cognitive Functions in any Personality Type on the Total Quantum of the Four Minor Cognitive Functions in any Personality Type; the result would be the Golden Number (1.6) or the range of the Golden Number (1.6−1.66666666667)=ϕ”. This result concluded and inspired from human body. When we divide the Essential or Main Part on the Minor Part, the result is 1.6. Example from: http://milan.milanovic.org/math/english/golden/golden2.html.

### QUANTITATIVE CONCEPT

The present invention depends on Mathematical—Physical Equilibrium:
1. Each Personality has a Total of Thinking (T)=Total of Feelings (F)=Total of Sensing (S)=Total Intuitive of (N)=100%. This means mathematically: (Te+Ti=100%), (Fe+Fi=100%), (Se+Si=100%) and (Ne+Ni=100%).
2. Thus there are 3 Levels of Quantum for a Cognitive Function; 25%, 50% and 75%. For the same Cognitive Function such as (T) which is Thinking or Logic Function, If the (Te)=75% then the (Ti)=25%, where bother (Te+Ti=75%+25%=100%) or (T=100%). A representation shows you the 3 Different Statuses for Te/Ti Quantum below. This Principle can be applied for All other Cognitive Function; Fe/Fi, Se/Si and Ne/Ni:
3. If the Cognitive Function is 75% in its Quantum, this means it is very Creative, Analytical and Masterminding. If the Cognitive Function is 50%, this means it is Coordinator, applier for Thoughts and Follows 75% Functions. If Cognitive is 25% which is inferior, it seeks learning for new things.
4. The Distribution of Quantum for each Cognitive Functions can be as Following:
   The 1^{st} and 2^{nd} Major/Essential Functions have to be 75% or at least one of them.
Any Major/Essential Function has to be 50% or 75%.
Any Minor/Assist Function has to be 25% or 50%.

From the above, you can Conclude 5 Models for (ESFP) Personality Types. This Variety in models Explains different Behaviors for (ESFP):

Method or Test to Define Accurate/Fine Personality Type

A very simple question can help an individual to discover their accurate Personality Types. The New Distinguish thing in Applicant's Model is; it refers to Quantum Energy of e/i=(Extroversion/Introversion) in Major Functions as well as Quantum Energy of j/p=(Judging/Perceiving) in Major Functions. Personality Types based on Major Functions include Questions in a Step after Defining your Personality Type by measuring the 16 Types for example.
1. What do you think of your Extroverted/Introverted Energy whether you're “E or I” Personality Type? (For Example: you can be very Enthusiastic Person with Introverted (I - - - ) Personality Type)
   Enthusiastic, Extroverted: ( ) e>i [symbol] j=p (Note: Don't think of (j) or (p) here) [symbol]
   Calm/Quiet, Introverted: ( ) i>e [symbol] j=p (Note: Don't think of(j) or (p) here)
   Balanced Level between Introverted and Extroverted: ( ) c=i [symbol] j=p or j>p or j<p
2. If you Chose third answer (e=i), what do you think of your Judging/Perceiving Energy whether you're “J or P” Personality Type?(For Example: you can be very organized Person with ( - - - P)ersonality)
   Extremely Organized and Acute: ( ) j>p, (Judging is higher than Perceiving)
   Balanced, half Strict and half Flexible: ( ) j=p, (Judging is balanced with Perceiving)
   Very Flexible and Adaptable with Situation: ( ) p>j, (Perceiving is Higher than Judging)

For example if you choose the First Option for (ESFP) in 1^{st} Question, you don't need to answer the 2^{nd} Question. Because if you have 2 pieces of information about (ESFP); e>1 and j=p which enables you to find the Accurate Type of (ESFP) among the 5 kinds. This Model is the First one among 5 (ESFP)s Models above. If you feel you are an (ESFP) with balanced level of Extroversion/Introversion and balanced level in Judging/Perceiving, then your Type is: (ESFP e=I j=p). This Model is third one between (ESFP) Models.

3. Medical Theories and Results:

(1) First Concept: Right-Left Brain Functions or (Brain-Split) which was discovered by Scientists Roger Wolcott, David Hunter Hubel and Torsten Nils Wiesel (Nobel Laureate). See Wikipedia References: (http://en.wikipedia.org/wiki/Roger_Wolcott_Sperry), (http://en.wikipedia.orglwiki/Cerebral_hemisphere). “Applicant applied this Result on the 8 Cognitive Functions. Applicant divided them into 2-Categories; Left Brain Category which is Logical, Sequential and Realistic one, will include Thinking Cognitive Functions (Te, Ti) and Sensory Cognitive Function (Se, Si). Then Right Brain Category which is Imaginative, Holistic and Emotional one, will include Feeling Cognitive Functions (Fe, Fi) and Intuitive (Ne, Ni)”. This Forms a Strong Connection or Link between Psychology (Personality Type) and Physiology! A Reference from: (http://www.biausa.org/living-with-brain-iniury.htm):

(2) Second Concept: Brain Wave and Neuron Electrical Activity that is measured by EEG Device. (See: http://en.wikipedia.org/wiki/Electroencephalography). From this Medical Concept, Applicant's Quantitative Concept defines Quantum as the Equivalent Number Corresponds to the Electrical Frequency of Human Brain Waves.

The 3 Numbers; 25%, 50%, 75% Form the Quantum of Major and Minor Cognitive Functions Personality Types models.
Then convert these 3 Numbers (7, 14, and 21) into a ratio by multiplying them with (3.571428571), which will give you (25, 50 and 75) to Correspond to Applicant's System for a Personality Type Model:

| Brain Waves in | Factor of | | Cognitive |
|---|---|---|---|
| Hz (Fixed | Conversion | New | Function |
| Numbers) | (Multiply) | Ratio | Quantum |
| 7 (Theta) | * 3.571428571 | 25 | 25% |
| 14 (Alpha) | * 3.571428571 | 50 | 50% |
| 21 (Beta) | * 3.571428571 | 75 | 75% |

| Hz | Medical Research | Quantum | Personality Proposal |
|---|---|---|---|
| 7 | Deep Relaxation, | 25% | Seek to learn, Inferior |
| | Low Conscious | | |
| 14 | Relaxed, Calm, Medium | 50% | Coordinators |
| | Conscious | | |
| 21 | Alert, Consciousness | 75% | Creative, Mastermind |

(1) Because Major Functions are Essential and Shape Personality Type, Applicant gives them Quantum 75%, and 25% for Minor Functions because they are Complementary for a Personality Type.

(2) From the above conclusion Alpha can be with Beta or Theta Ranges!” which leads Applicant to classify Alpha wave as a middle Quantum and equal to 50%. This Quantum can be for Majors or Minors Functions:
From (1) and (2); Applicant could summarize the Three different Quantum: (25%, 50% and 75%) for Major/Minor Functions as Following:

| Cognitive Programming | Quantum Probability |
|---|---|
| Minor Functions | 25% or 50% |
| Major Functions | 75% or 50% |

These Numbers are Correspond to Applicant's System; All 8 Cognitive Functions are founded in an Equal Quantum (T=F=S=N=100%) but they are founded in Different Distribution or Programming for each Personality Type based on its Cognitive Function (Major/Minor):

(1) Thinking (T) is a System equals to (Te+Ti=100%) and can be take 3 Kinds of Energy Transformations like: When (Te=75% [symbol] Ti=25%), When (Te=50% [symbol] Ti=50%), and Finally When (Te=25% [symbol] Ti=75%).

(2) Feeling (F) is a System equals to (Fe+Fi=100%) and can be take 3 Kinds of Energy Transformations like: When (Fe=75% [symbol] Fi=25%), When (Fe=50% [symbol] Fi=50%), and Finally When (Fe=25% [symbol] Fi=75%).

(3) Sensing (S) is a System equals to (Se+Si=100%) and can be take 3 Kinds of Energy Transformations like: When (Se=75% [symbol] Si=25%), When (Se=50% [symbol] Si=50%), and Finally When (Se=25% [symbol] Si=75%).

(4) Intuition (N) is a System equals to (Ne+Ni=100%) and can be take 3 Kinds of Energy Transformations like: When (Ne=75% [symbol] Ni=25%), When (Ne=50% [symbol] Ni=50%), and Finally When (Ne=25% [symbol] Ni=75%).

When (T)=Thinking or Logic, (F)=Feeling or Emotions, (S)=Sensing or Reality, (N)=Intuitive or Imagination, Then: (T+F) is Making Decisions System & (N+S) is Absorbing Information.

First: If Quantum of Thinking (T) in any Personality is more than Quantum of Feeling (F), this means the Personality is like a Computer . . . No benefits for using Feeling because (T) is dominating, or T>F. So the State: T>F (is not Correct) . . . (1.1)

And If Quantum of Feeling (F) in any Personality is more than Quantum of Thinking (T), this means the Personality is Subjective and makes All decisions based on Feeling System . . . and this is abnormal. So the State: F>T (is not Correct) . . . (1.2)

Now, if we propose that: (T) is Parallel to (F) or (T) is Balanced or in Equilibrium with (F), then you can say: (T) is equal in Quantum with (F) or (F=T) where the Makes Decisions of Human Thinking System is balanced or in Equilibrium Now! So The State: T=F (is Correct) . . . (1.3)

From above:

(F+T)=Making Decision System, and (F=T):(Quantum of Feeling=Quantum of Thinking)

Now, we can propose a Value of F and T. Say F=T=I or F=T=100% which is easy for calculation; division and measurement, etc. And this is the First Result!

Second: If Quantum of Intuition (N) in any Personality is more than Quantum of Sensing (S), this means the Personality is basically a Dreamer . . . Detached from others and Can't Understand Physical Events or Interaction between others because (N) is more than (S) or (N) is dominating or say: N>S. So the State: N>S (is not Correct) . . . (2.1)

And If Quantum of Sensing (S) in any Personality is more than Intuition (N), this means the Personality Can't understand any New or Innovative idea, or say can't develop things because the mind is trapped with Physical Interactions . . . And this is abnormal Situation when (S) is domination. So the State: S>N (is not Correct) . . . (2.2)

Now, if: (N) is parallel to (S) or (N) is balanced or in Equilibrium with (S), then: (N) is equal in Quantum with (S) or (N=S) where the Information System of Human Thinking is balanced or in Equilibrium Now! So the State: N=S (is Correct) . . . (2.3)

From above:

(N+S)=Information System, and (N=S):(Quantum of Intuition=Quantum of Sensing)

Now, we can propose a Value of F and T. Say N=N=1 or N=N=100% which is easy for calculation; division and measurement, etc. And this is the Second Result!

Now, We have to Proof why both of (T=F)=100% and (N=S)=100% or say why (T=F=N=S)=100%?

Third: If we propose: (T=F)>(N=S) or Quantitatively (T=F)>100% and (N=S)=100%, then a Person tends to Make Decisions more than Absorbing Information and this is abnormal. If we proposed (N=S)>(T=F) or Quantitatively (N=S)>100% and (T=F)=100%, then a Person is always receiving or absorbing information without making benefits of them because the making decisions system is Quantitatively less than information system, so the information are NOT treated or filtered well and this is abnormal case and logically not correct! So both of States: (T=F)>(N=S) & (N=S)>(T=F) are NOT Correct!

If we proposed that the Total Quantum of Information System (N+S) is Equal to the Total Quantum of Decisions Making System (T+F), then the Overall System of Human Thinking is Balanced and Work Well. So the State: (T=F)=(N=S) is Correct and Equal to 100% ! And this is the Third Result!

Now, if we combine First Result: (T=F=100%), Second Result: (N=S=100%) and Third one: (T=F)=(N=S)=100%, then:

(T=F=N=S)=100% (Rule)

Now Apply the above Rule Qualitatively on (Split-Brain Research) or say (Right-Left Functions) which states: The Right Brain is Imaginative and Emotional where The Left Brain is Logical and Realistic:

This means Right Brain will include: (N+F); intuitive and Feeling or (Ne+Ni+Fe+Fi) where Left Brain will include: (T+S); Thinking and Sensing or (Te+Ti+Se+Si) and would be Distributed as the Following:

Now, let's Apply the above Rule: (T=F=N=S=100%) Quantitatively on (Split-Brain Research) or (Right-Left Functions):

Total Quantum=T+S+N+F=400%

### SUMMARY

1. 3-Fields, All together Form a basic Concept:
   Psychological or Personality Types
   Medical Theory (Right/Left) Brain
   Mathematical—Physical Equilibrium
2. Personality Behavior:
   The Structural Model (Order of Cognitive Functions; 1^{st}, 2^{nd}, 3^{rd} and 4^{th}) is one for ENTJ, ESTJ, ENFJ and ESFJ:
3. Balance and Integration Relationships between Cognitive Functions:
   The following is an Example of, ESTJ/ENTJ; the Cognitive Function. The Cognitive Function (Te) is Balanced with (Fe), because (Te) is in the Left Brain and (Fe) in the Right Brain. Also (Te) integrates with (Ti) to have a better understanding about Logic or Thinking (T) for FSTJ/ENTJ.
4. From Quantitative (Quantum) Concept; one reaches the following Results:
   a. (ST)=Sensor-Thinkers Group: can't be Right Brain (R.B), but only Whole (W.B) or Left (L.B) Brain.
   b. (NF)=Intuitive-Feelers Group: can't be Left Brain (L.B), but only Whole (W.B) or Right (R.B) Brain.
   c. (SF)=Sensor-Feelers Group: can be Right Brain (R.B), Left (L.B) and Whole (W.B) Brain.
   d. (NT)=Intuitive-Thinkers Group: can be Right Brain (R.B), Left (L.B) and Whole (W.B) Brain.
5. In general the Major Functions with (75%) Quantum determines the behavior of Personality:
6. Cognitive Cycle in New Model:
   Consider an Example (ESFP e>i j=p):

As you can see from the (ESFP) Model above, there are two Loops; (Extraverted Loop “e-Loop”) is Upper Loop and (Introverted Loop “i-Loop”) is Lower Loop:

Upper Loop has a high chance/opportunity to use, and

Lower Loop has a Low chance/opportunity to use.

In General:
When you are Introverted, you concentrate on “i” Functions or using “i-Loop”,
When you are Extroverted, you concentrate on “e” Functions or using “e-Loop”.

In Model above:
The “e-Loop Cycle” in order (Se [symbol] Te [symbol] Fe [symbol] Ne) then [symbol] (Se) again.
The “i-Loop Cycle” in order: (Fi [symbol] Ni [symbol] Si [symbol] Ti) then [symbol] (Fi) again.

However the Normal Cycle/Frequency for (ESFP) is: (Se+Te) then (Fi+Ni).

(ESFP) Links and Connections: [symbol]
(Se+Te) [symbol] is a “Major Link” with “Extraverted Connection (e)”
(Fi+Ni) [symbol] is a “Major Link” with “Introverted Connection (i)”
(Fe+Ne) [symbol] is a “Minor Link” with “Extraverted Connection (e)”
(Si+Ti) [symbol] is a “Minor Link” with “Introverted Connection (i)
7. Scale of Extroversion/Introversion and Judging/Perceiving:

Indicator

Now consider the indicators of 16 Pages, each Personality in a Separated Page. Each Page is divided into 3 Parts:
The First/Top Part is “Model and General Analysis about a Personality Type”, which concentrates on Cognitive Connections and Programming.
The Second/Middle Part is “Fine Analysis on the 5 Models of a Personality Type”, which concentrates on discovering a Personality Type Behavior based on Right, Left and Whole Brain.
Third/below Part is “Cognitive Cycle of a Personality Type”.

The First and Third Parts can be applied for All 5 Personalities in Second Part.

How can you know you have done the Correct Choice in Method/Test?

Very Easy, Example you are Logical and Realistic person i.e. Left Brain (LB) Person but the result in the model is Right Brain (R.B) which is Creative, Holistic and Emotional person! This Contradiction should direct you to repeat the Test/Method. The Target of the test is to discover quantum of extroversion/introversion (e/i) as well as quantum of judging/perceiving (j/p) in Major Functions which assists in Right, Left and Whole Brain Analysis.

Application

To determine if the proposal is correct consider the following Example of Famous Scientific Portraits; (Albert Einstein) and (Nikola Tesla). Both of them are (INTP) but each one of them has different Personality than the other. Applicant chose these Personalities because they are Famous and you can search about them, their Scientific Inventions as well as their Personal Lives the Final Result with Analysis follow.

From (Nikola Tesla) Model: (INTP i>e j=p):

As Personal Level: Tesla is very Calm, Quiet and Sensitive and this is True in his Model because i>e. Also Tesla didn't get married, he dislikes social life which reflect his introverted energy more than extroverted one. Tesla has few close friends.

As Scientific Invention: Tesla is an Electrical and Mechanical Engineer and Inventor. His works depends on Logic, Realistic and Practical Visions, Tools and Technical things. He has High of (Ti+Si) Energy because Engineering needs Logical (T) and Sensory (S) as Cognitive Functions more than holistic and Imagination. Also Tesla said one day, he wants to Invalidate “General Relativity” of Einstein which means that he has an opposite thinking with Einstein, and both of them are INTP.

From (Albert Einstein) Model: (INTP e>i j=p):

As Personal Level: Einstein is completely Normal Person in Social Level/Marriage and Very Good in Communications with People and sometimes rude with them because e>i, NOT like Tesla Sensitive and Shy with people because i>e. (i=Quantum of Major Introverted Functions, and e=Major Extroverted Functions).

As Scientific Invention: Einstein is a Philosopher and Physicist. He is Theoretical and Imaginative. This appears in “General Relativity” which needs a Person with a High Level of Flexibility and Imagination to understand that thing; the Fabric of Space-time. Also “Special Relativity” needs Imagination, Idea of Conversion between Mass and Energy. This is easy for Einstein because he has High Quantum of Intuitive and Feeling (Ne+Fe).

### INDEX AND REFERENCES

1. 8-Cognitive Functions:

http://www.cognitiveprocesses.com/Cognitive-Functions/index.cfm

1. Thinking (T):

Extraverted Thinking (Te)
Contingency planning, scheduling, and quantifying utilize the process of extraverted Thinking.
Extraverted Thinking helps us organize our environment and ideas through charts, tables, graphs, flow charts, outlines, and so on.
At its most sophisticated, this process is about organizing and monitoring people and things to work efficiently and productively.
Empirical thinking is at the core of extraverted thinking when we challenge someone's ideas based on the logic of the facts in front of us or lay out reasonable explanations for decisions or conclusions made, often trying to establish order in someone else's thought process.
In written or verbal communication, extraverted Thinking helps us easily follow someone else's logic, sequence, or organization.
It also helps us notice when something is missing, like when someone says he or she is going to talk about four topics and talks about only three.
In general, it allows us to compartmentalize many aspects of our lives so we can do what is necessary to accomplish our objectives.

Introverted Thinking (Ti)
Introverted Thinking often involves finding just the right word to clearly express an idea concisely, crisply, and to the point.
Using introverted Thinking is like having an internal sense of the essential qualities of something, noticing the fine distinctions that make it what it is and then naming it.
It also involves an internal reasoning process of deriving subcategories of classes and sub-principles of general principles.
These can then be used in problem solving, analysis, and refining of a product or an idea.
This process is evidenced in behaviors like taking things or ideas apart to figure out how they work.
The analysis involves looking at different sides of an issue and seeing where there is inconsistency.
In so doing, we search for a “leverage point” that will fix problems with the least amount of effort or damage to the system.
We engage in this process when we notice logical inconsistencies between statements and frameworks, using a model to evaluate the likely accuracy of what's observed.

2. Feeling (F):

Extraverted Feeling (Fe)
The process of extraverted Feeling often involves a desire to connect with (or disconnect from) others and is often evidenced by expressions of warmth (or displeasure) and self-disclosure.
The “social graces,” such as being polite, being nice, being friendly, being considerate, and being appropriate, often revolve around the process of extraverted Feeling.
Keeping in touch, laughing at jokes when others laugh, and trying to get people to act kindly to each other also involves extraverted Feeling.
Using this process, we respond according to expressed or even unexpressed wants and needs of others.
We may ask people what they want or need or self-disclose to prompt them to talk more about themselves.
This often sparks conversation and lets us know more about them so we can better adjust our behavior to them.
Often with this process, we feel pulled to be responsible and take care of others' feelings, sometimes to the point of not separating our feelings from theirs.
We may recognize and adhere to shared values, feelings, and social norms to get along.

Introverted Feeling (Fi)
It is often hard to assign words to the values used to make introverted Feeling judgments since they are often associated with images, feeling tones, and gut reactions more than words.
As a cognitive process, it often serves as a filter for information that matches what is valued, wanted, or worth believing in.
There can be a continual weighing of the situational worth or importance of everything and a patient balancing of the core issues of peace and conflict in life's situations.
We engage in the process of introverted Feeling when a value is compromised and we think, “Sometimes, some things just have to be said.”
On the other hand, most of the time this process works “in private” and is expressed through actions.
It helps us know when people are being fake or insincere or if they are basically good. It is like having an internal sense of the “essence” of a person or a project and reading fine distinctions among feeling tones.

3. Sensing (S):

Extraverted Sensing (Se)
Extraverted Sensing occurs when we become aware of what is in the physical world in rich detail.
We may be drawn to act on what we experience to get an immediate result.
We notice relevant facts and occurrences in a sea of data and experiences, learning all the facts we can about the immediate context or area of focus and what goes on in that context.
An active seeking of more and more input to get the whole picture may occur until all sources of input have been exhausted or something else captures our attention.
Extraverted Sensing is operating when we freely follow exciting physical impulses or instincts as they come up and enjoy the thrill of action in the present moment.
An oneness with the physical world and a total absorption may exist as we move, touch, and sense what is around us.
The process involves instantly reading cues to see how far we can go in a situation and still get the impact we want or respond to the situation with presence.

Introverted Sensing (Si)
Introverted Sensing often involves storing data and information, then comparing and contrasting the current situation with similar ones.
The immediate experience or words are instantly linked with the prior experiences, and we register a similarity or a difference—for example, noticing that some food doesn't taste the same or is saltier than it usually is.
Introverted Sensing is also operating when we see someone who reminds us of someone else.
Sometimes a feeling associated with the recalled image comes into our awareness along with the information itself. Then the image can be so strong, our body responds as if reliving the experience. The process also involves reviewing the past to draw on the lessons of history, hindsight, and experience.
With introverted Sensing, there is often great attention to detail and getting a clear picture of goals and objectives and what is to happen. There can be a oneness with ageless customs that help sustain civilization and culture and protect what is known and long-lasting, even while what is reliable changes.

Intuiting (N):

Extraverted Intuiting (Ne)
Extraverted Intuiting involves noticing hidden meanings and interpreting them, often entertaining a wealth of possible interpretations from just one idea or interpreting what someone's behavior really means.
It also involves seeing things “as if,” with various possible representations of reality.
Using this process, we can juggle many different ideas, thoughts, beliefs, and meanings in our mind at once with the possibility that they are all true.
This is like weaving themes and threads together.
We don't know the weave until a thought thread appears or is drawn out in the interaction of thoughts, often brought in from other contexts.
Thus a strategy or concept often emerges from the here-and-now interactions, not appearing as a whole beforehand.
Using this process we can really appreciate brainstorming and trust what emerges, enjoying imaginative play with scenarios and combining possibilities, using a kind of cross-contextual thinking.
Extraverted Intuiting also can involve catalyzing people and extemporaneously shaping situations, spreading an atmosphere of change through emergent leadership.

Introverted Intuiting (Ni)
Introverted intuiting involves synthesizing the seemingly paradoxical or contradictory, which takes understanding to a new level.
Using this process, we can have moments when completely new, unimagined realizations come to us.
A disengagement from interactions in the room occurs, followed by a sudden “Aha!” or “That's it!” The sense of the future and the realizations that come from introverted intuiting have sureness and an imperative quality that seem to demand action and help us stay focused on fulfilling our vision or dream of how things will be in the future.
Using this process, we might rely on a focal device or symbolic action to predict, enlighten, or transform.
We could find ourselves laying out how the future will unfold base on unseen trends and telling signs.
This process can involve working out complex concepts or systems of thinking or conceiving of symbolic or novel ways to understand things that are universal.
It can lead to creating transcendent experiences or solutions.

B. Producing Models of a Personality Type from All Previous Rules:

Summary of All Rules Concluded from All Previous Processes:

1. Golden Ratio: Φ
:(Related to 16 Personality Types and Human Body Members Divisions: Major Part/Minor one)

$$\frac{Total Quantum of Magor Cognitive Functions}{Total Quantum of Minor Cognitive Functions} = Φ = 1.6$$

2. Major—Minor Cognitive Functions
:(From Brain Wave or Neuron Electrical Activity)

| Cognitive Programming | Quantum Probability |
|---|---|
| Major Functions | 75% or 50% |
| Minor Functions | 25% or 50% |

3. Quantitative-Equilibrium
:(Mathematical-Physical Representation of Brain Energy Equilibrium and Transformnnation)

3.1 All Cognitive Functions are Equal: (Te+Ti)=(Fe+Fi)=(Ne+Ni)=(Se+Si)=100% Then: (T==N=S)=100% [symbol] Cognitive Function Balanced

3.2 Total Quantum of Making Decisions=Total Quantum of Receiving Information Then: (T+F)=(N+S)=200% [symbol] Human Thinking System Balanced

[symbol]

Total Quantum of Left Brain (L.B)=Total Quantum of Right Brain (R.B) Then: (T+S)=(N+F)=200% [symbol] Left-Right Brain Balanced

Now, How can we conclude a Personality Model from Previous Rules?

I will use Trial-Error method to solve this Problem.

If we take our Example (ESFP), the New Qualitative Distribution is:

Now, How can we Distribute Quantum (25%, 50%, 75%) on that model?

We know that Major Functions can be two values: 50% or 75% while Minor Functions: 50% or 25%. Now the Probability of Quantitative Distribution on previous model would like the Following:

Now we will have 2 Probability for each 8 Functions, and By using Trial-Errors Method, We can check which models could validate the previous Rules? No. (1); Golden Ratio and No. (3); Mathematical-Physical Equilibrium of Cognitive Functions and their Energies Transformation?

(Note: (/) means canceling this Number or Value):

Analysis:

If we apply the Golden Ratio Rule here; Σ Major/Minor=(50+50+50+50)/(50+50+50+50)=1≠1.6 (ϕ) Then [symbol] this is not accepted model because it does not validate Rule No. (1); Golden Ratio.

### Model (1) is Rejected

Analysis:

If we apply the Golden Ratio Rule here; Σ Major/Σ Minor=(75+75+75+75)/(25+25+25+25)=3≠1.6 (ϕ) Then [symbol] this is not accepted model because this model does not validate Rule No. (1); Golden Ratio.

### Model (2) is Rejected

Analysis:

(1) If we apply the Golden Ratio Rule here; Σ Major/Σ Minor=(50+50+75+75)/(50+50+25+25)=1.6 (ϕ) Then [symbol] this model validates Rule No. (1); Golden Ratio.

(2) Total of Right Brain: (25+75+25+75)=Total of Left Brain: (50+50+50+50)=200%, and S=N=F=T=100%. Then [symbol] this model validates Rule No. (3); Mathematical-Physical Equilibrium.

### Model (3) is Accepted

Analysis:

(1) If we apply the Golden Ratio Rule here; Σ Major/Σ Minor=(75+75+50+50)/(50+50+25+25)=1.6 (ϕ) Then [symbol] this model validates Rule No. (1); Golden Ratio.

(2) Total of Right Brain: (50+50+50+50)=Total of Left Brain: (25+75+25+75)=200%, and S=N=F=T=100%. Then [symbol] this model validates Rule No. (3); Mathematical-Physical Equilibrium.

### Model (4) is Accepted

Analysis:

Note that; in Right Brain; (Fi+Fe)=(50+25)=75% 100%. Then [symbol] this model doesn't validate Rule No. (3) especially (3.1).

### Model (5) is Rejected

Analysis:

Note that; in Right Brain; (Fi+Fe)=(75+50)=125%≠100%. Then [symbol] this model doesn't validate Rule No. (3) especially (3.1).

### Model (6) is Rejected

Analysis:

(1) If we apply the Golden Ratio Rule here; Σ Major/Σ Minor=(75+75+50+50)/(50+50+25+25)=1.6 (ϕ) Then [symbol] this model validates Rule No. (1); Golden Ratio.

(2) Total of Right Brain: (50+50+75+25)=Total of Left Brain: (50+75+25+50)=200%, and S=N=F=T=100%. Then [symbol] this model validates Rule No. (3); Mathematical-Physical Equilibrium.

[symbol]

### Model (7) is Accepted

Analysis:

Left Brain Quantum=(75+50+75+50)=250% where Right Brain Quantum=(75+50+75+50)=250%. This is not validating condition/rule No. (3): Σ R.B=Σ L.B=200% and NOT 250% ! Where the total of All Quantum=400% and not 500%.

### Model (8) is Rejected

Analysis:

Left Brain Quantum=(25+50+25+50)=150% where Right Brain Quantum=(25+50+25+50)=150%. This is not validating condition/rule No. (3): because Σ R.B=Σ L.B=200% and NOT 150% ! Where the total of All Quantum=400% and not 300%.

### Model (9) is Rejected

Analysis:

Right Brain Quantum=(50+75+50+75)=250%, Then not validating condition No. 3, Σ L.B should be 200%!

### Model (10) is Rejected

Comment on Models:

As you can see from previous models it's hard to conclude All Correct Models that validate the 3 conditions. What we need is to extract the common factors between the 3 accepted models.

Now let's Study the 3 Successful models:

Conclusion from 3 Accepted Models:

1. Major Functions Values in All 3 models are: (75%, 75%, 50% and 50%) where Minor Functions Values are: (50%, 50%, 25%, and 25%) which validates Rule No. (1); Σ Majors/Σ Minor=(75+75+50+50)/(50+50+25+25)=(250/150)=1.66 (Φ) (Golden Ratio This means; There is a determinate Value for total Major Functions=250% and total Minor=150%.

Total of Higher Functions (Se+Ne+Ti+Fi)=200%=Total of Lower Functions (Te+Fe+Si+Ni) or you can say: Σ Higher/Σ Lower=200/200=1. This Result makes our Model Quantitatively Symmetric:

Total Higher Major Functions (Se+Fi) is greater than or equal to Total Lower Major Functions (Te+Ni) or you can say: Σ Higher Major≥Σ Lower Major. This means the Probability Values of Total Higher Major can be (75+75)=150% as a Maximum Value or (75+50)=125% as a Minimum Value and cannot be can (50+50)=100% because Σ Lower Major≥Σ Higher Major. How did I reach this? Or . . . Why Total of Higher Major Functions is greater than or equal to the Total of Lower Major Functions?

Let's take this Example:

Note that:
(1) Σ Major/Σ Minor=(50+75+50+75)/(25+50+25+50)=(250/150)=1.6=Phi (Golden Ratio)
(2) (T=F=S=N)=100%
(3) Σ R.B=Σ L.B=200/200=1
(4) All 3 Conditions are Correct but: Σ Higher/Σ Lower=150/250=0.6 and NOT 1 (which is not Correct) based on previous conclusion. But if we consider this is a correct result: Σ Lower could be Greater than Σ Higher, then it is Qualitatively NOT Correct. Why?

Because this will contradict with Natural Behavior of ESFP in (SF) Group which tends to be easy-going, entertainer and good parents . . . etc. Note that both of (Te) and (Ni) are Lower Major Functions, but their value is 75%. This means ESFP is like ENTJ in (NT) or Rational Group which considers to be out of ESFP Nature; Realistic and Physically Interactive especially through (Se) Function and Fun-Lover, Entertainer and Kind-Hearted through (Fi) Cognitive Function. This means in All ESFP models; the Quantum of (Se) and/or (Fi) should be at Least 75%, which means Result No. 3 in last Conclusion is Correct; which states “ . . . the Probability Values of Total Higher Major can be (75+75)=150% as a Maximum Value or (75+50)=125% as a Minimum Value and cannot be can (50+50)=100%”. This Result Also Confirm and Validate Result No. (2): Σ Higher/Σ Lower=200/200=1. Now what are other models for ESFP? I need to re-arrange all Quantitative Results in nice clear Format.

Final Quantitative Conclusion:
1. In Higher Functions: The Value of two Higher Major Functions: =(75% and 75%) or (50% and 75%) where Lower Major Functions: =(75% and 50%) or (50% and 50%) which means The Total of Higher Major Functions is greater than or equal to Total of Lower Major Functions Or. Σ Higher Major≥Σ Lower Major
2. All Cognitive Functions are Equal: (Te+Ti)=(Fe+Fi)=(Ne+Ni)=(Se+Si)=100% Or: (T=F=N=S)=100% [symbol] Cognitive System Equilibrium or Balance:
3. Total Quantum of Making Decisions=Total Quantum of Receiving Information Or: (T+F)=(N+S)=200% [symbol] Human Thinking System Equilibrium or Balance
4. Total Quantum of Left Brain (L.B)=Total Quantum of Right Brain (R.B) Or: (T+S)=(N+F)=200% [symbol] Left-Right Brain Equilibrium or Balance
5. Total of Four Higher Functions is Equal to the Total of Four Lower Functions; Or: Σ Higher/Σ Lower=200/200=1
6. Total Quantum of the Four Major Functions divided by Total Quantum of the Four Minor Functions equal to Golden Ratio or Golden Number or Range of Phi (1.6−1.666667), which can be only expressed when Total Major Quantum=250% and Total Minor=150%: Σ Major/Σ Minor=(50+75+50+75)/(25+50+25+50)=(250/150)=1.66=Phi

Now by synthesize the above Developed Results with each other in a Creative way and by using Trial-and-Error Method, We will have Two New Models for ESFP here:

Check-Up and Analysis:

Model (4):
(1) Σ Higher Major=(Se+Fi)=(75+75)=150% and, Σ Lower Major=(Te+Ni)=(50+50)=100%. Then our Rule: Σ Higher Major≥Σ Lower Major is Correct!
(2) (Te+Ti)=(Fe+Fi)=(Ne+Ni)=(Se+Si)=100% [symbol] Cognitive System Equilibrium is Correct!
(3) (T+F)=(N+S)=200% [symbol] Human Thinking System Equilibrium or Balance is Correct!
(4) (T+S)=(N+F)=200% [symbol] Left-Right Brain Equilibrium or Balance is Correct!
(5) Σ Higher=(Se+Fi+Ne+Ti)=(75+75+50+50)=250% where: Σ Lower=(Te+Fe+Ni+Si)=(50+25+50+25)=150%
   Then our Rule: Σ Higher≥Σ Lower is Correct!
(6) Σ Major/Σ Minor=(Se+Fi+Te+Ni)/(Ne+Ti+Fe+Si)=(75+75+50+50)/(50+50+25+25)=(250/150)=1.66=Phi [symbol] Golden Ratio is Correct!

Model (5):
(1) Σ Higher Major=(Se+Fi)=(75+50)=125% and, Σ Lower Major=(Te+Ni)=(50+75)=125%. Then our Rule: Σ Higher Major≥Σ Lower Major is Correct!
(2) (Te+Ti)=(Fe+Fi)=(Ne+Ni)=(Se+Si)=100% [symbol] Cognitive System Equilibrium is Correct!
(3) (T+F)=(N+S)=200% [symbol] Human Thinking System Equilibrium or Balance is Correct!
(4) (T+S)=(N+F)=200% [symbol] Left-Right Brain Equilibrium or Balance is Correct!
(5) Σ Higher=(Se+Fi+Ne+Ti)=(75+50+25+50)=200% where: Σ Lower=(Te+Fe+Ni+Si)=(50+50+75+25)=200%
   Then our Rule: Σ Higher≥Σ Lower is Correct!
(6) Σ Major/Σ Minor=(Se+Fi+Te+Ni)/(Ne+Ti+Fe+Si)=(75+50+50+75)/(25+50+50+25)=(250/150)=1.66=Phi [symbol] Golden Ratio is Correct!

[symbol]

Conclusion

Summarizing All ESFP Models:
1. I conclude any 5 models for any Personality Type by following the same previous procedures because All Types must share the same Conditions. With this Scenario, Total Number of All Personality Types Models is: 5*16=80. Before you Conclude any Personality Types Models Quantitatively, you have to Distribute All Cognitive Functions on Right and Left brain Correctly as well as Distributing them in Higher or Lower brain Correctly, and which Function is Major or Minor for this Personality Type which I mean “The Qualitative Model”.
2. Note How can I represent ESFP Model? We have two Options:

Analyzing the 5 Models of One Personality Types (Example): ESFP

1. The Accurate Quantum of (F/I) and (P/J) in a Personality Type
Model:(The 5 Statuses) and (Right/Left/Balanced Behavior Analysis):

Explain Cognitive Cycles (e/i)

First: Quantum of E/I and P/J in a Personality Types (The 5 Statuses):

In any Model, we have Four Extraverted Functions: (Se, Te, Ne and Fe) and Four Introverted Functions: (Fi, Ni, Ti and Si). In Carl Jung Theory, we can realize that; there is a Specific Behavior for Each Cognitive Function. For Example (T and F) Functions such as (Te, Ti, Fe and Fi) have Judger Behavior where (N and S) Functions such as (Ne, Ni, Se and Si) have Perceiver Behavior.

So I can Summarize Carl Jung Theory in that way:

When you apply any Quantitative Model on Jung Theory, You can discover Quantum of E/I and J/P. The important here is we'll take the Major Functions only in our Consideration because they Shape the Real Character of the Personality Type.

Let's Apply that on ESFP models:

If you calculate the Major Perceiving Function: (Se+Ni)=50+75=125%, then The Major Judging Functions: (Fi+Te)=75+50=125%. Note: they are Equal to each other 125%! This means the Quantum of Perceiving is equal to Quantum of Judging or P=J in . . . (1)

Now, calculate the Extraversion and Introversion of Major Functions. Extraverted Functions: (Se+Te)=50+50=100%, and the Introverted Functions: (Fi+Ni)=75+75=150% which means the Quantum of Introversion is Greater than Quantum Extraversion or mathematically, I>E . . . (2)

Analyzing Behavior and Rename Models:

Now if you combine result (1) and (2) for model (1) [symbol] ESFP (P=J), (I>E) or in other way to rename this model, we can make the letters (P, J, I and E) small letter. Then the model will be like [symbol] ESFP (p=j), (i>e)

Note the order between (p=j) and (i>e) is not important here, which means we can write this model like this way: [symbol] ESFP (i>e), (p=j)

Note that the in Model (1), Ni and Fi are Major Functions and They have the Highest Quantum which is 75%. This means these two Cognitive Functions are important in this model or say takes the Largest Area of Thinking or Say Dominating, which leads me to innovate new name for this model: [symbol] ESFP_{(Ni,Fi)}

This name is easy to use for All because the main distinguishing Functions for ESFP (Ni, Fi) represent Right Brain Personality; Very Creative and discover things in a Fresh-Eye because (Ni) is dominating and also it's an Emotional Personality very Sensitive when dealing with People through very High Standards and Principles because (Fi) is dominating here. Also you can Conclude that this Personality is Shy and Calm when Dealing with People because Quantum of Introversion is Greater than Quantum Extraversion (i>e); even it's an Extraverted Personality ESFP which begins with (Se) Extraverted Cognitive Functions. This Personality has the lowest level of Extraversion or Highest Level of Introversion even it is extraverted one (E - - - ).

Major Perceiving Function: (Se+Ni)=75+50=125%, and Major Judging Functions: (Fi+Te)=50+75=125%, which means: Quantum of P=Quantum of J or: [symbol] P=J . . . (1)

Major Extraverted Functions: (Se+Te)=75+75=150%, and Major Introverted Functions: (Fi+Ni)=50+50=100%, which means: Quantum of E>Quantum of I or: [symbol] E>I . . . (2)

From (1) and (2), ESFP model (2) can be expressed as:

[symbol]

ESFP(P=J),(E>I) or ESFP(p=j),(e>i) or ESFP_{(Se,Te)}

Analyzing Behavior:

With (Se) and (Te) dominating this Personality is more into Left Brain Logical and Realistic one. This Personality is very good at interactive with Physical Events because (Se) is dominating and very good at dealing with things logically because (Te) is dominating. Also it has the Highest Level of Extraversion because both of (Se) and (Te) are Extraverted Functions which means this personality is Excellent when dealing with People with Super Communication Skills.

Major Perceiving Function: (Se+Ni)=50+50=100%, and Major Judging Functions: (Fi+Te)=75+75=150%, which means: Quantum of J>Quantum of P or: [symbol] J>P . . . (1)

Major Extraverted Functions: (Se+Te)=50+75=125%, and Major Introverted Functions: (Fi+Ni)=75+50=125%, which means: Quantum of E=Quantum of I or: [symbol] E=I . . . (2)

From (1) and (2), ESFP model (3) can be expressed as:

[symbol]

ESFP(J>P),(E=I) or ESFP(j>p),(e=i) or ESFP_{(Fi,Te)}

Analyzing Behavior:

With (Fi) and (Te) dominating this Personality is more into Whole Brain or Balanced Brain, Half Right and Half Left. Personality is Emotional and Logical or Say Executive to apply Emotional Principles because (Fi) is dominating and also doing the right things because (Te) is dominating. This Personality has a Normal Level of Extraversion-Introversion because their dominating Major Functions is Extraverted which is (Te) and other one is Introverted which is (Fi) which make this Personality Balanced when dealing or interacting with People or outer World.

Major Perceiving Function: (Se+Ni)=75+5( )=125%, and Major Judging Functions: (Fi+Te)=75+50=125%, which means: Quantum of P=Quantum of J or: [symbol] P=J . . . (1)

Major Extraverted Functions: (Se+Te)=75+50=125%, and Major Introverted Functions: (Fi+Ni)=75+50=125%, which means: Quantum of E=Quantum of I or: [symbol] E=I . . . (2)

From (1) and (2), ESFP model (4) can be expressed as:

[symbol]

ESFP (P=J),(E=I) or ESFP(p=j),(e=i) or ESFP_{(Se,Fi)}

Analyzing Behavior:

With (Se) and (Fi) dominating, this Personality is Balanced between Left Brain and Right Brain or says Balanced or Whole Brain. This Personality is Realistic with Excellent Physical Interaction with Outer Environment (Events and Things) because the Cognitive Function (Se) is dominating here. At the same time (Fi) is dominating here which means Emotional System (Inner Principles) takes a Large Area for this Personality. This Type is balanced when dealing with people i.e. the level of Extraversion-Introversion is balanced here.

Major Perceiving Function: (Se+Ni)=75+75=150%, and Major Judging Functions: (Fi+Te)=50+50=100%, which means: Quantum of P>Quantum of J or: [symbol] P>J . . . (1)

Major Extraverted Functions: (Se+Te)=75+50=125%, and Major Introverted Functions: (Fi+Ni)=50+75=125%, which means: Quantum of E=Quantum of I or: [symbol] E=I . . . (2)

With this two piece of informnnation (1) and (2), ESFP model (5) can be expressed as:

[symbol]

ESFP (P>J),(E=I) or ESFP(p>j),(e=i) or ESFP_{(Se,Ni)}

Analyzing Behavior:

With (Se) and (Ni) dominating this Personality is whole or balanced between Left Brain and Right Brain. This Personality has strong Sensation abilities because (Se) is dominating as well as Strong Intuitive Vision to see connection between things and meaning because (Ni) dominating. This personality is very Flexible and Creative and can be founded in many fields; Artistic or Scientific one. The Level or Scale of Extraversion—Introversion is medium or say balanced when dealing with People or outer Environment.

Level or Scale of (E/I) and (P/J)

From above process, you can conclude beauty of Personality Type, the new thing we discover here is; the Accurate or Fine Behavior of a Personality; like the introverted behavior inside Extraverted Personality. You can notice there are three levels of Extroversion and Introversion or say three (E/I) Scales I reached when I deal with Extraverted Personality like ESFP:

And they are: (E - - - ) (e>i) or (e=i) or (i>e)

And also there are three levels of Judging and Perceiving for Extraverted Personality like ESFP:

And they are: (E - - - ) (p>j) or (p=j) or (p<j)

Now, if my theory is balanced, the Introverted Personality (I - - - ) should also has three levels of (E/I) and (P/J). If you apply same process in (Page 21, 22, 23 and 24) on any Introverted Personality (I - - - ), you will have the same result: (F/I) Scale: (I - - - ) (e>i) or (e=i) or (i>e) and (P/J) Scale: (I - - - ) (p>j) or (p=j) or (p<j).

But can we integrate All Scales together for Extraverted and Introverted Personalities?

Qualitative Principle:

The Extraverted Personality (E - - - ) which begins with Extraverted Cognitive Function is more Active and more opened into the outer world and it deals with people more than Introverted Personality (I - - - ) that is more likely into discovering the inner or hidden meanings between things. This means All Three Levels of Extraverted Personality is higher in their Levels of Extraversion than the Three Levels of Introverted Personality Type which can expressed as the Following:

However I note that there is a Strong approximation between Highest Level of Extraversion in Introverted Personality: (I - - - )_{(c>i)} and Lowest Level of Extraversion in Extraverted Personality:

(E - - - )_{(i>e)} which makes me think to put them parallel to each other in Second Proposal in Next Chart. Both of Proposals are true, and the Second is more accurate.

The same method applied on (E/I) Scale can also be applied on (J/P) Scale to end-up Finally with this Chart:

General Behavior for a Personality inside a Group

ESFP shares the same Qualitative Model with ESTP, ENTP and ENFP. Quality: is the same order of Cognitive Functions (1^{st}, 2^{nd}, 3^{rd} and 4^{th}) in Right or Left Brain. However the Programming of Major and Minor Functions is only the Different here. This Quanlitative Structure leads me to Classify the 16 Personality Types into 4 behaviours inside their Groups:

The 4 Groups are:
1. Sensor Feelers (—SF—): Fun-Lover, Kind-hearted, Entertainer, Easy-going and Friendly Parents.
2. Intuitive Feelers (—NF—): Art Directors, Etiquette Interest, Love Peace and Creative Parents.
3. Sensor Thinkers (—ST—): Like Logical Operations, Applications and Appreciate Justice.
4. Intuitive Thinkers (—NT—): Tends to Strategy, Science, Inventions to discover new things.

The following pages provide specific guides for the analysis of 16 psychological types.

While the invention has been described in connection with its preferred embodiments, it should be recognized that changes and modifications may be made therein without departing from the scope of the appended claims.

1-20. (canceled)

21: A method of assessing a personality type of an individual, comprising the steps of:
measuring alpha, beta and theta brainwaves of an individual using an electroencephalogram as the individual performs a Myers-Briggs personality type test;
determining a Myers-Briggs personality type indicator from the Myers-Briggs personality type test;
determining a personality sub-type based on a predominant one of the measured alpha, beta and theta brainwaves of the individual, wherein a predominance of measured theta brainwaves indicates at least a personality which seeks to learn, a predominance of alpha brainwaves indicates at least a personality which tends to coordinate, and a predominance of beta brainwaves indicates at least a personality which is creative; and
combining the Myers-Briggs personality type indicator and the personality sub-type to determine an overall personality type and sub-type associated with the individual.
